(19) United States
(12) Patent Application Publication
(10) Pub. No.: US 2022/0142554 A1
(43) Pub. Date: May 12, 2022

(54) SYSTEM AND METHOD FOR MONITORING NEURAL SIGNALS

(71) Applicant: The General Hospital Corporation, Boston, MA (US)

(72) Inventors: Patrick L. Purdon, Somerville, MA (US); Hugo Soulat, Boston, MA (US); Amanda M. Beck, Boston, MA (US); Emily P. Stephen, Boston, MA (US)

(21) Appl. No.: 17/259,659

(22) PCT Filed: Jul. 16, 2019
(86) PCT No.: PCT/US19/42082

§ 371 Date: Jan. 12, 2021

Related U.S. Application Data
(60) Provisional application No. 62/698,435, filed on Jul. 16, 2018.

(51) Int. Cl. A61B 5/374; A61B 5/291; A61B 5/384

(52) U.S. Cl. CPC A61B 5/374; A61B 2560/02; A61B 5/384; A61B 5/291

(57) ABSTRACT

In accordance with one aspect of the disclosure, a method for estimating at least one oscillatory component of a patient brain state is provided. The method includes receiving electroencephalogram (BEG) data, fitting a space state model to the EEG data, the state space model comprising at least one oscillatory component, estimating at least one value of cross-frequency coupling of the EEG data based on the state space model, generating a report based on the at least one value of cross-frequency coupling, and outputting the report to at least one of a display and a memory.

### CROSS REFERENCE TO RELATED APPLICATIONS

This application is based on, claims priority to, and incorporates herein by reference in its entirety, U.S. Provisional Application Ser. No. 62/698,435, filed Jul. 16, 2018, and entitled “System and Method for Monitoring Neural Oscillations.”

### STATEMENT REGARDING FEDERALLY SPONSORED RESEARCH

This invention was made with government support under R01AG056015-01, R01AG054081-01A1, and GM118269-01A1 awarded by the National Institutes of Health. The government has certain rights in the invention.

### BACKGROUND

Oscillations in neural signals are ubiquitous and reflect the coordinated activity of neuronal populations across a wide range of temporal and spatial scales. Neural oscillations are understood to play a significant role in mediating many aspects of brain function, including attention, cognition, sensory processing, and consciousness. Accordingly, neurocognitive and psychiatric disorders, as well as altered states of consciousness, have been associated with altered or disrupted brain oscillations. Neural oscillations can be recorded in both animal and human models, at scales spanning individual neuron membrane potentials, neural circuits, and non-invasive scalp electromagnetic fields. Thus, neural oscillations can provide a valuable mechanistic scaffold to link observations across different scales and models.

Unfortunately, neural signals are quite subtle when compared to many other physiological signals and can be obscured, completely or partially, by a myriad of influences. As such, robust signal processing and/or signal discrimination it is often a precursor to any attempt to acquire and/or analyze neural signals.

For example, brain oscillations are often analyzed using frequency domain methods, such as nonparametric spectral analysis, or time domain methods based on linear bandpass filtering. A typical analysis might seek to estimate the power within an oscillation sitting within a particular frequency range, for instance, an alpha oscillation whose frequency is between 8 and 12 Hz. Often, the power between 8 to 12 Hz is computed in frequency domain using the power spectrum, or in time domain by estimating the power or variance in a bandpass filtered signal.

Processing of this nature is predicated upon a variety of assumptions, some of which may be made in error and others of which may be made based on conscious tradeoffs. For example, the above-described frequency selection and filtering inherently discriminates against some “signal” in an effort to remove “noise.” However, the portions of the “signal” that is lost with the “noise” may be more valuable than assumed. Furthermore, many signal processing techniques, such as bandpass frequency filtering, are readily available to implement, but may not be the ideal signal processing technique to best capture the “signal.”

Thus, it would be desirable to have systems and methods that better facilitate acquisition and analysis of neural signals.

### SUMMARY

The present disclosure provides systems and methods for processing neural signals relative to oscillatory states, such as for use in patient monitoring and de-modulation of signals in applications beyond brain monitoring. In one non-limiting aspect, the present disclosure provides system and method for capturing neural signals as a combination of linear oscillatory representations within a state space model. The parameters of these models can use an expectation maximization algorithm or other methods to select an appropriate model, which serves to identify the oscillations present in the data and, thereby, extract the desired neural signal. With proper extraction such as provided by these systems and methods, the present disclosure is able to distinguish or separate the oscillations of interest from potential secondary physiology signals that would otherwise confound subsequent analyses. As such, the systems and methods of the present disclosure are able to estimate properties of the oscillations that are useful for monitoring applications, such as phase and amplitude cross correlation, phase of an oscillation, oscillatory amplitude, oscillatory power, relative oscillatory power, oscillatory bandwidth, oscillatory resonance, oscillatory quality-factor, or the like.

In accordance with one aspect of the disclosure, a method for estimating at least one oscillatory component of a patient brain state is provided. The method includes receiving electroencephalogram (EEG) data, fitting a space state model to the EEG data, the state space model comprising at least one oscillatory component, estimating at least one value of cross-frequency coupling of the EEG data based on the state space model, generating a report based on the at least one value of cross-frequency coupling, and outputting the report to at least one of a display and a memory.

In the method, the at least one value of cross-frequency coupling can include an estimated value of phase amplitude modulation between a first wave component and a second wave component included on the state space model.

In the method, the first wave can be a slow wave component and the second wave can be an alpha wave component.

In the method, the estimated value of phase amplitude modulation can be calculated based on a phase of the slow wave component and an amplitude of the alpha wave component.

In the method, the at least one value of cross-frequency coupling can include a correlation value between an oscillatory component included in the state space model and a band of frequencies of the EEG data.

In the method, the fitting the state space model can include fitting harmonic frequencies of each oscillatory component included in the at least one oscillatory component.

In the method the at least one oscillatory component can include an alpha component, and the alpha component is associated with prior distribution for a center frequency. The prior distribution can be a Von Mises prior.

In the method, a damping factor of the state space model can be constrained with a prior distribution. The prior distribution can be a beta distribution.

In accordance with another aspect of the disclosure, a system for estimating at least one oscillatory component of a patient brain state is provided. The system includes a plurality of sensors configured to receive electroencephalogram (EEG) data, a processor configured to receive the EEG data and carry out steps including fitting a space state model to the EEG data, the state space model comprising at least one oscillatory component, estimating at least one value of cross-frequency coupling of the EEG data based on the state space model, generating a report based on the at least one value of cross-frequency coupling, and a display configured to display the report.

In the system, the at least one value of cross-frequency coupling can include an estimated value of phase amplitude modulation between a first wave component and a second wave component included on the state space model.

In the system, the first wave can be a slow wave component and the second wave can be an alpha wave component.

In the system, the estimated value of phase amplitude modulation can be calculated based on a phase of the slow wave component and an amplitude of the alpha wave component.

In the system, the at least one value of cross-frequency coupling can include a correlation value between an oscillatory component included in the state space model and a band of frequencies of the EEG data.

In the system, the fitting the state space model can include fitting harmonic frequencies of each oscillatory component included in the at least one oscillatory component.

In the system, the at least one oscillatory component can include an alpha component, and the alpha component is associated with prior distribution for a center frequency. The prior distribution can be a Von Mises prior.

In the system, a damping factor of the state space model can be constrained with a prior distribution. The prior distribution can be a beta distribution.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A shows an embodiment of a physiological monitoring system 10

FIG. 1B shows an exemplary EEG sensor.

FIG. 2 shows an exemplary system for patient monitoring.

FIG. 3 shows an exemplary process for acquiring data and generating a report.

FIG. 4 shows another exemplary process for acquiring data and generating a report.

FIG. 5A shows the EEG spectrum for an autoregressive model in a person receiving propofol.

FIG. 5B shows the EEG spectrum for an autoregressive model during a resting state.

FIG. 6A shows a combined oscillation time series.

FIG. 6B shows a slow oscillation time series of FIG. 6A.

FIG. 6C shows the alpha oscillation time series of FIG. 6A.

FIG. 7 shows multitaper spectra for bandpass slow and alpha components.

FIG. 8A shows a total signal for a bandpass method and a state space method.

FIG. 8B shows a slow oscillation component for a bandpass method and a state space method.

FIG. 8C shows an alpha oscillation component for a bandpass method and a state space method.

FIG. 8D shows residuals for a bandpass method and a state space method.

FIG. 9A shows spectra of AR components of EEG data.

FIG. 9B shows spectra of estimated oscillations of the EEG data.

FIG. 10A shows spectra of AR components for a slow and alpha model without a Von Mises prior.

FIG. 10B shows spectra of estimated oscillations for the slow and alpha model without a Von Mises prior.

FIG. 10C shows spectra of AR components for a slow without alpha model.

FIG. 10D shows spectra of estimated oscillations for the slow without alpha model.

FIG. 11 shows an exemplary process 1100 for fitting EEG data with a state space model.

FIG. 12A shows raw EEG data.

FIG. 12B shows a six second window of the EEG data.

FIG. 12C shows a slow oscillation fit in the six second window.

FIG. 12D shows a fast oscillation fit in the six second window.

FIG. 12E shows an exemplary form of the state space model.

FIG. 12F shows a slow oscillation phase with credible intervals.

FIG. 12G shows a fast oscillation amplitude with credible intervals.

FIG. 12H shows an estimated K^{mod} and associated distribution.

FIG. 12I shows an estimated ϕ^{mod} and associated distribution.

FIG. 12J shows regressed alpha wave amplitude.

FIG. 13A shows a first fit pass for a parametric power spectral density data for given data.

FIG. 13B shows a threshold for the parametric power spectral density data.

FIG. 13C shows a second fit pass for parametric power spectral density data.

FIG. 13D shows a redressed power spectral density data.

FIG. 13E shows a fit line pass for parametric power spectral density data.

FIG. 14A shows a propofol concentration supplied to a patient EEG data.

FIG. 14B shows a response probability corresponding to the patient EEG data.

FIG. 14C shows modulation regression values of the patient EEG data.

FIG. 14D shows a parametric spectrum of the patient EEG data.

FIG. 14E shows a modulogram PAM of the patient EEG data.

FIG. 14F shows modulation parameters of the patient EEG data.

FIG. 15A is a response probability plot.

FIG. 15B is a propofol concentration plot.

FIG. 15C is a modulogram for a standard approach with six second time windows.

FIG. 15D is a modulation index plot associated with FIG. 15C.

FIG. 15E is a modulogram for a standard approach with one hundred and twenty second time windows.

FIG. 15F is a modulation index plot associated with FIG. 15E.

FIG. 15G is a modulogram for the SSCFA approach.

FIG. 15H is a modulation index plot associated with FIG. 15G.

FIG. 15I is a modulogram for the dSSCFA approach.

FIG. 15J is a modulation index plot associated with FIG. 15I.

FIG. 16A is a response probability plot. FIG. 16B is a propofol concentration plot.

FIG. 16C is a modulogram for a standard approach with six second time windows.

FIG. 16D is a modulation index plot associated with FIG. 16C.

FIG. 16E is a modulogram for a standard approach with one hundred and twenty second time windows.

FIG. 16F is a modulation index plot associated with FIG. 16E.

FIG. 16G is a modulogram for the SSCFA approach.

FIG. 16H is a modulation index plot associated with FIG. 16G.

FIG. 16I is a modulogram for the dSSCFA approach.

FIG. 16J is a modulation index plot associated with FIG. 16I.

FIG. 17A shows decomposition of components of oscillatory components using a prior method.

FIG. 17B shows power spectral density determined using the prior method of FIG. 17A.

FIG. 17C shows a phase amplitude cross correlation estimate by the prior method of FIG. 17A.

FIG. 17D shows decomposition of components of oscillatory components using a SSCFA method.

FIG. 17E shows power spectral density determined using the SSCFA method of FIG. 17D.

FIG. 17C shows a phase amplitude cross correlation estimate by a prior method.

FIG. 17F shows a phase amplitude cross correlation estimate by the SSCFA method of FIG. 17F.

FIG. 18A shows dynamics of amplitude modulation.

FIG. 18B shows dynamics of phase modulation.

FIG. 19A shows correlation based cross-frequency coupling for a frontal electrode.

FIG. 19B shows a combined modulogram summary.

FIG. 20A shows cross-frequency coupling principal frequency modes as a function of frequency.

FIG. 20B shows percentages of total energy for the principal frequency modes.

FIG. 21 shows projected cross-frequency coupling patterns onto a first principal frequency mode.

FIG. 22 shows a graph of broadband frequency coupling by low frequency activity at different locations of the brain during different states projected onto the first principal frequency mode.

FIG. 23 shows an exemplary process for performing cross-frequency coupling analysis using fitted state space models.

### DETAILED DESCRIPTION

As will be described herein, an example patient monitoring system and methods of use are provided. As one non-limiting example, as will be described, the system includes sensors that can be used to provide physiological monitoring of a patient, such as monitoring of a patient experiencing an administration of at least one drug having anesthetic properties.

For example, as will be described, one non-limiting application for the systems and methods is to monitor general anesthesia, sedation, or other drug-induced states. Thus, in accordance with one aspect of the disclosure, a system for monitoring of a patient experiencing an administration of at least one drug having anesthetic properties is provided. The system can include a plurality of sensors configured to acquire physiological data from the patient while receiving at least one drug having anesthetic properties and at least one processor configured to acquire physiological data from the plurality of sensors, and determine, from the physiological data, signal markers that can be used to determine the patient's brain state during general anesthesia or sedation or other drug-induced state.

The systems and methods may also provide closed-loop control of drug administration to produce and regulate brain states required for general anesthesia, sedation, or other drug-induced states. Thus, in accordance with one aspect of the disclosure, a system for closed-loop control of a patient experiencing an administration of at least one drug having anesthetic properties is provided. The system can include a plurality of sensors configured to acquire physiological data from the patient while receiving the at least one drug having anesthetic properties and at least one processor configured to acquire physiological data from the plurality of sensors, and determine, from the physiological data, signal markers that can be used to infer the patient's brain state during general anesthesia or sedation or other drug-induced state. The system can also include drug delivery systems to administer drugs based on the brain state information provided by the monitor.

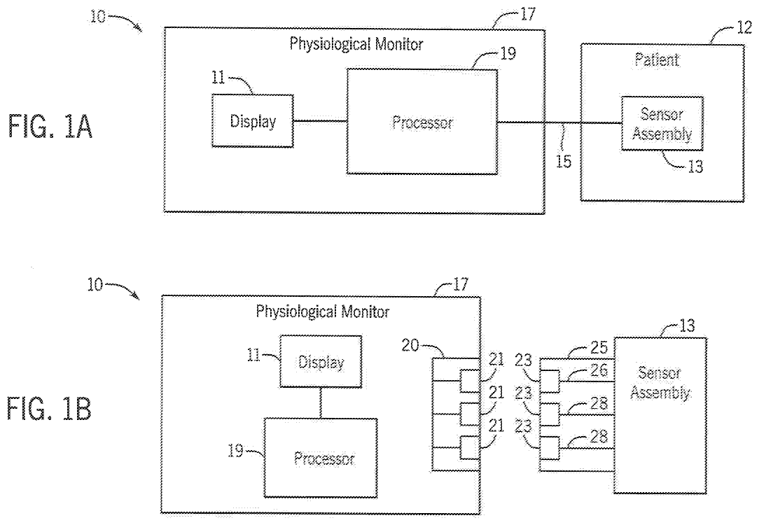

Referring now to the drawings, FIG. 1A shows an example of a physiological monitoring system 10. In the physiological monitoring system 10, a medical patient 12 is monitored using one or more sensors 13, each of which transmits a signal over a cable 15 or other communication link or medium to a physiological monitor 17. The physiological monitor 17 includes a processor 19 and, optionally, a display 11. The one or more sensors 13 include sensing elements such as, for example, electrical EEG sensors, or the like. The sensors 13 can generate respective signals by measuring a physiological parameter of the patient 12. The signals are then processed by one or more processors 19. The one or more processors 19 then communicate the processed signal to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

For clarity, a single block is used to illustrate the one or more sensors 13 shown in FIG. 1A. It should be understood that the sensor 13 shown is intended to represent one or more sensors. In an embodiment, the one or more sensors 13 include a single sensor of one of the types described below. In another embodiment, the one or more sensors 13 include at least two EEG sensors. In still another embodiment, the one or more sensors 13 include at least two EEG sensors and one or more brain oxygenation sensors, and the like. In each of the foregoing embodiments, additional sensors of different types are also optionally included. Other combinations of numbers and types of sensors are also suitable for use with the physiological monitoring system 10.

In some configurations of the system shown in FIG. 1A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors 13.

As shown in FIG. 1 B, the EEG sensor 13 can include a cable 25. The cable 25 can include three conductors within an electrical shielding. One conductor 26 can provide power to a physiological monitor 17, one conductor 28 can provide a ground signal to the physiological monitor 17, and one conductor 28 can transmit signals from the sensor 13 to the physiological monitor 17. For multiple sensors, one or more additional cables 15 can be provided.

In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor 13 and the physiological monitor 17 communicate wirelessly.

In some configurations, systems shown in FIGS. 1A and 1B may further include a memory, database or other data storage locations (not shown), accessible by processor 19, to include reference information or other data. Specifically, such reference information can include a listing of patient categories, such as various age categories, along with associated signals, signal markers or signatures. For example, signal markers or signatures can include various signal amplitudes, phases, frequencies, power spectra, or ranges or combinations thereof, as well as other signal markers or signatures. In some aspects, such reference information can be used by the processor 19 to determine specific patient characteristics, such an apparent or likely patient age, or other patient conditions or categories. In other aspects, data acquisition may be regulated or modified based on selected and/or determined patient characteristics.

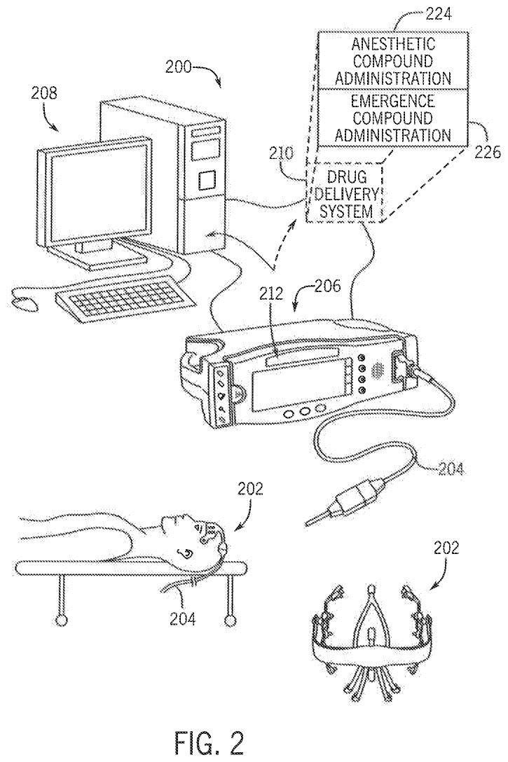

Specifically now referring to FIG. 2, an exemplary system 200 in accordance with aspects of the present disclosure is illustrated, which may be constructed as a stand-alone brain monitoring device, or portable device, or could be incorporated as a central component of an existing brain monitoring device. As will be appreciated from forthcoming descriptions, the system 200 may find valuable usage within an operating room or an intensive care setting, in association with conducting a variety of medical procedures, such as during administration of an anesthetic, as well as within a pre- or post-operative evaluation situation.

The system 200 includes a patient monitoring device 202, such as a physiological monitoring device, illustrated in FIG. 2 as an electroencephalography (EEG) electrode array. However, it is contemplated that the patient monitoring device 202 may include a number of different sensors. In particular, the patient monitoring device 202 may also include mechanisms for monitoring galvanic skin response (GSR), for example, to measure arousal to external stimuli or other monitoring system such as cardiovascular monitors, including electrocardiographic and blood pressure monitors, and also ocular microtremor monitors. One realization of this design may utilize a frontal Laplacian EEG electrode layout with additional electrodes to measure GSR and/or ocular microtremor. Another realization of this design may incorporate a frontal array of electrodes that could be combined in post-processing to obtain any combination of electrodes found to optimally detect the EEG signatures described earlier, also with separate GSR electrodes. Another realization of this design may utilize a high-density layout sampling the entire scalp surface using between 64 to 256 sensors for the purpose of source localization, also with separate GSR electrodes.

The patient monitoring device 202 is connected via a cable 204 to communicate with a monitoring system 206. Also, the cable 204 and similar connections can be replaced by wireless connections between components. The monitoring system 206 may be configured to receive raw signals from patient monitoring device 202, such as signals acquired by the EEG electrode array, and assemble, process, and even display the signals in various forms, including time-series waveforms, spectrograms, and the like. In some modes of operation, the monitoring system 206 may be designed to acquire scout data, in the form of physiological or other data, from sensors on the patient monitoring device 202 and identify, using the scout data, signal markers, or signatures therein. For example, signal amplitudes, phases, frequencies, power spectra, and other signal markers or signatures, may be identified in scout data, and other acquired data, using various suitable methods. In addition, a multitaper analysis may be performed to identify and account for a dynamic range of signals spanning several orders of magnitude. Such signal markers or signature may then be used by the monitoring system 206 to determine various patient characteristics, including an apparent and/or likely patient age.

In one embodiment, acquisition of physiological data using monitoring system 206 may be adjusted or regulated based patient characteristics determined from scout data. Specifically, the monitoring system 206 may be configured to determine a scale consistent with certain determined patient characteristics, and adjust subsequent data acquisition, based on the determined scale and/or any indication provided by user. For instance, data acquisition may be regulated by adjusting one or more amplifier gains, along with other data acquisition parameters. Moreover, in some aspects, the monitoring system 206 may be further configured to format various acquired physiological data to be displayed against the scale. In this manner, an age-appropriate scale may be determined based on the apparent and/or likely patient age, and any subsequent data acquisition using a selected age-appropriate scale would generate and illustrate age-compensated data.

As illustrated, the monitoring system 206 may be further connected to a dedicated analysis system 208. However, the monitoring system 206 and analysis system 208 may be integrated or combined into a common system. The analysis system 208 may receive EEG waveforms from the monitoring system 206 and, as will be described, analyze the EEG waveforms and signatures therein. However, it is also contemplated that any analysis or processing functions of the monitoring system 206 and analysis system 208 may be shared or individually distributed, as required or desired.

In some aspects, information related to determined characteristics of a patient undergoing a specific medical procedure may be provided to a clinician or operator of system 200. For example, it was previously found that elderly patients were more likely to enter burst suppression in the operating room. Specifically, burst suppression is the profound state of brain inactivation in which bursts of electrical activity are interspersed with isoelectric periods termed suppressions. Brain states of anesthetic-induced unconsciousness, defined by the alpha wave (8-10 Hz) and slow wave (0.1-4 Hz) signal oscillations, can be obtained with doses of anesthetics that are less than those required to produce burst suppression. This may mean reducing anesthetic dosing to levels substantially less than what are currently recommended for elderly individuals. Because currently recommended doses typically place elderly patients into burst suppression, adequate states of general anesthesia and reduced anesthetic exposure may be achievable by titrating anesthetic dosing based on real-time EEG monitoring. Hence system 200 may provide, based on determined patient characteristics, information for use in selecting an appropriate anesthetic dosing. In this manner, for example, incidence of post-operative cognitive disorders for elderly patients under general anesthesia may be reduced.

In another example, monitoring system 206 and/or analysis system 208 may be capable of providing a pre- or post-operative assessment of specific patients, such as the young, middle-aged and elderly, as well as drug addicted patients, to determine prior information that could be used to identify and/or predict specific patient conditions, including anesthetic sensitivity, and any potential for post-operative complications, such as cognitive disorders. Moreover, specific regimens for anesthetic care, post-anesthesia care, or intensive care, may also be provided.

The system 200 may also include a drug delivery system 210. The drug delivery system 210 may be coupled to the analysis system 208 and monitoring system 208, such that the system 200 forms a closed-loop monitoring and control system. Such a closed-loop monitoring and control system in accordance with the present disclosure is capable of a wide range of operation, but includes user interfaces 212 to allow a user to configure the closed-loop monitoring and control system, receive feedback from the closed-loop monitoring and control system, and, if needed reconfigure and/or override the closed-loop monitoring and control system. In some configurations, the drug delivery system 210 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness.

For example, in accordance with one aspect of the present invention, methylphenidate (MPH) can be used as an inhibitor of dopamine and norepinephrine reuptake transporters and actively induces emergence from isoflurane general anesthesia. MPH can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. Also, ethylphenidate or other agents can be used to actively induce emergence from isoflurane, propofol, or other general anesthesia by increasing arousal using a control system, such as described above.

Therefore, a system, such as described above with respect to FIG. 2, can be provided to carry out active emergence from anesthesia by including a drug delivery system 210 with two specific sub-systems. As such, the drug delivery system 210 may include an anesthetic compound administration system 224 that is designed to deliver doses of one or more anesthetic compounds to a subject and may also include a emergence compound administration system 226 that is designed to deliver doses of one or more compounds that will reverse general anesthesia or the enhance the natural emergence of a subject from anesthesia.

For example, MPH and analogues and derivatives thereof induces emergence of a subject from anesthesia-induced unconsciousness by increasing arousal and respiratory drive. Thus, the emergence compound administration system 326 can be used to deliver MPH, amphetamine, modafinil, amantadine, or caffeine to reverse general anesthetic-induced unconsciousness and respiratory depression at the end of surgery. The MPH may be dextro-methylphenidate (D-MPH), racemic methylphenidate, or leva-methylphenidate (L-MPH), or may be compositions in equal or different ratios, such as about 50 percent:50 percent, or about 60 percent:40 percent, or about 70 percent:30 percent, or 80 percent:20 percent, 90 percent:10 percent,95 percent:5 percent and the like. Other agents may be administered as a higher dose of methylphenidate than the dose used for the treatment of Attention Deficit Disorder (ADD) or Attention Deficit Hyperactivity Disorder (ADHD), such as a dose of methylphenidate can be between about 10 mg/kg and about 5 mg/kg, and any integer between about 5 mg/kg and 10 mg/kg. In some situations, the dose is between about 7 mg/kg and about 0.1 mg/kg, or between about 5 mg/kg and about 0.5 mg/kg. Other agents may include those that are inhaled.

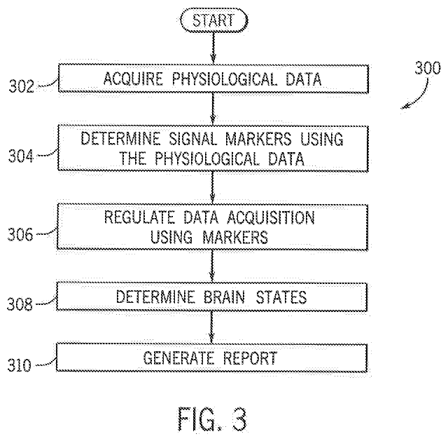

Turning to FIG. 3, a process 300 in accordance with aspects of the present disclosure is shown. Beginning with process block 302, any amount of physiological data may be acquired, wherein the physiological data is representative of physiological signals, such as EEG signals, obtained from a patient using, for example, the patient monitoring device 202. In some aspects, the physiological data may include scout data for purposes including determining various patient characteristics. Then at process block 304, signal markers or signatures are identified or determined using the acquired physiological data. For example, signal amplitudes, phases, frequencies, power spectra, and other signal markers or signatures, may be identified in scout data, and/or other acquired data, using various suitable methods.

In some preferred embodiments, the signal markers or signatures may be used to determine patient characteristics, including an apparent and/or likely patient age. In addition, process block 304 may also include steps of determining a scale consistent with determined patient characteristics. In one aspect, use of spectral estimation methods, such as the multi-taper method, that can inherently account for a wide dynamic range of signals spanning many orders of magnitude may be employed. In another aspect, an automatic estimation of signal amplitudes may be performed to infer a correct age cohort and attendant settings for a visualization scale, as well as for acquisition amplifier gains.

At the next process block 306, using the signal markers or signatures determined from the scout data, a data acquisition process may be adjusted or regulated, in relation to signal data to be acquired subsequently. For instance, data acquisition may be regulated by adjusting one or more amplifier gains, along with other data acquisition parameters. In some aspects, regulating data acquisition may also include using a scale consistent with determined patient characteristics, and adjusting the subsequent data acquisition based on the determined scale and/or any indication provided by user. By way of example, an age-appropriate scale determined at process block 304, based on the apparent and/or likely patient age, may be used, and any subsequent data acquisition using a selected age-appropriate scale would generate age-compensated data.

At process block 308, data acquired in a manner described may be used to determine current or future brain states of patient. For example, analyzed or processed EEG waveforms assembled using age-compensated data may be used to assess a present and/or future depth of anesthesia or sedation. In addition, determining such brain states may also include any information provided by a clinician or user, such as information related to a medical procedure.

Then at process block 310 a report is generated, for example, in the form a printed report or, preferably, a real-time display. The report may include raw or processed data, signature information, indications of current or future brain states, as well as information related to patient-specific characteristics, including as a likely and/or apparent patient age. Displayed signature information or determined states may be in the form of a waveforms, spectrograms, coherograms, probability curves and so forth. In some aspects, the report may include formatted physiological data displayed against a scale. In other aspects, the report may indicate an anesthetic sensitivity, a probability for post-operative complications, such as cognitive disorders, and also regimens for anesthetic care, post-anesthesia care, or intensive care, and so forth

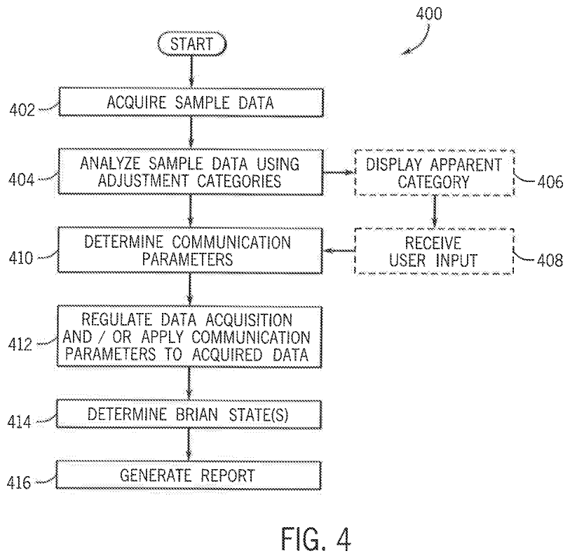

Turning to FIG. 4, steps of another process 400 in accordance with aspects of the present disclosure are illustrated. Specifically, the process 400 begins at process block 402 where sample or scout data is acquired using, for example, patient monitoring systems, as described. At process block 404, the sample data is then analyzed using adjustment or reference categories, to identify patient categories representative of the acquired sample data. Specifically, this step can include identifying signal markers or signatures in the sample data and performing a comparison with signal markers or signatures associated with the reference categories. For example, signal amplitudes, phases, frequencies, power spectra, and other signal markers or signatures, can be detected in the sample data using various suitable methods.

Analysis, as performed at process block 404, can indicate specific patient characteristics, including an apparent and/or likely patient age. In some aspects, an identified or apparent category indicating specific patient characteristics may be optionally displayed at process block 406. Moreover, at process block 408 a user input may also be received.

Subsequently, at process block 410 a determination is made with respect to various communication parameters. This includes taking into consideration determined or inferred patient characteristics or categories, and optionally a user input. For example, an age-appropriate scale for the acquired data may be determined at process block 410 based on determined patient characteristics and/or signals, signal markers or signatures present in the acquired data. Then at process block 412, a subsequent data acquisition may be regulated using the determined communication parameters to acquire age-appropriate data. As described, regulating data acquisition may include appropriately adjusting or modifying various amplifier gains using the communication parameters. In some aspects, the determined communication parameters may be directly applied to the acquired sample data. For example, an age-appropriate scale may be applied to the sample data to create age-appropriate data.

Then, at process block 414, data acquired or processed in a manner described may be used to determine current or future brain states of patient. For example, analyzed or processed EEG waveforms assembled using age-compensated data may be used to assess a present and/or future depth of anesthesia or sedation. In addition, determining such brain states may also include any information provided by a clinician or user, such as information related to a medical procedure.

Then at process block 416 a report is generated of any suitable shape or form. In some aspects, the report may display scaled data or data categories describing the data. In other aspects, the report may indicate an anesthetic sensitivity, a probability for operative or post-operative complications, an apparent or likely patient age, and other information related to aspects of the present disclosure.

There are a number of ways in which the present systems and methods can be used to provide indicators of brain state that can be used to assess neural signals to derive indicators or other information that is useful in patient monitoring. One such non-limiting example may include:

Determining and/or analyzing characteristics of amplitudes of slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

Determining and/or analyzing characteristics of analyzing power within slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like

Determining and/or analyzing characteristics of the peak frequency of an oscillation, including slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

Determining and/or analyzing characteristics of coherence between sensors, or among a plurality of sensors, for slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

Determining and/or analyzing characteristics of the phase of slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

Determining and/or analyzing characteristics of phase differences between sensors or between groups of sensors, for slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

Determining and/or analyzing characteristics of cross-frequency coupling relationships between multiple oscillations, for example relationships between the phase of one oscillator and the amplitude of the other, including slow oscillations (0.1 to 1 Hz), delta oscillations (1 to 4 Hz), theta oscillations (4 to 8 Hz), alpha oscillations (8 to 12 Hz), beta oscillations (12 to 25 Hz), gamma oscillations (25 to 70 Hz), and high-gamma oscillations (70 Hz and above), or the like.

In one example, to monitor and track general anesthesia or sedation, the peak frequency of an oscillation spanning the theta (4 to 8 Hz), alpha (8 to 12 Hz), and beta bands (12 to 25 Hz) could be presented. Alongside this, the power in the slow oscillation (0.1 to 1 Hz) and delta (1 to 4 Hz) bands could be presented. These specific indicators, provided by the system and method presented here, can be used, for example, by an anesthesiologist or other clinician to discern states of general anesthesia corresponding to slow/delta and alpha oscillations, distinct from a state of sedation featuring beta and gamma oscillations, when an anesthetic drug such as propofol or sevoflurane are administered.

In another example, to monitor and track general anesthesia or sedation, the phase-amplitude coupling between the phase of a slow (0.1 to 1 Hz) through delta (1 to 4 Hz) oscillation and the amplitude of an alpha (8 to 12 Hz) through beta (12 to 25 Hz) oscillation can be presented. The phase of that relationship can be used to distinguish between a sedative state (for example, “troughmax” modulation) or a state of deep unconsciousness (for example, “peakmax” modulation). The strength of the coupling relationship could be used to regulate, under closed-loop control, the amount of anesthetic to deliver to maintain a deep state of unconsciousness.

In another example, the present system and method can be used to represent secondary physiology signals distinct from the physiological signals of interest, such as the EEG and EEG-derived quantities. For instance, a low-frequency first-order autoregressive AR(1) component can be added to the state dynamics component of the model to account for low-frequency drifts. Similarly, a state-space model of movement-induced secondary physiology waveforms could also be included. Similarly, an AR or moving-average component can be incorporated in the state dynamics component of the model to represent muscle -induced secondary physiology signals. In this way, the presented system and method can be used to separate secondary physiology signals from the physiological signals of interest. These secondary physiology signals may be useful, in and of themselves, as indicators of different states, such as consciousness, or movement.

In another mode of operation, the system can use information from other devices, such as other physiological monitors, or from the medical record system, to inform the interpretation of the brain or physiological state recorded from EEG or physiological sensors. This information can inform the use of specific oscillator models as well as specific secondary physiology models to provide the desired monitoring variables.

In another mode of operation, the system can employ a database of models and model parameterizations meant to represent different clinical scenarios. The system can use information from the medical record, such as patient demographic information or medical history, as well as information about anesthetic drugs administered, as well as information about the type of surgical case being performed, to select a set of candidate models that might apply to a given scenario. The system can use the EEG or other physiologic data recorded in the specific patient being monitored to select the model or models that best apply in a given scenario. The database of models can take many different forms, including prior probability distributions of different parameters, represented as functions of different input variables, or as in the form of look-up tables, or similar concepts.

Generally, the oscillations of an EEG can be represented as distinct components that are observed in noise. For example, a slow and an alpha oscillation under propofol anesthesia can be modeled using the below equation:

yₜ=x_{slow,t}+x_{alpha,t}+vₜ (1)

A more general case is described below. Each oscillatory component can then be modeled using a second-order autoregressive (AR(2)) process, which can be written in state space form. It is noted that the model of each oscillatory component is technically autoregressive moving average (ARMA) models due to the presence of observation (which will be described below), but we will refer to the models according to their underlying state dynamics to emphasize the oscillatory structure of the model. It can be assumed, for the moment, that the observed signal yₜ∈ [symbol] is a linear combination of two latent states representing a slow and a fast component xₜ^{s} and xₜ^{f}∈ [symbol] ², respectively. Each of these 2-dimensional (2D) latent states can be assumed to be independent and their evolution over a fixed step size is modeled as a scaled and noisy rotation, for j=s, f:

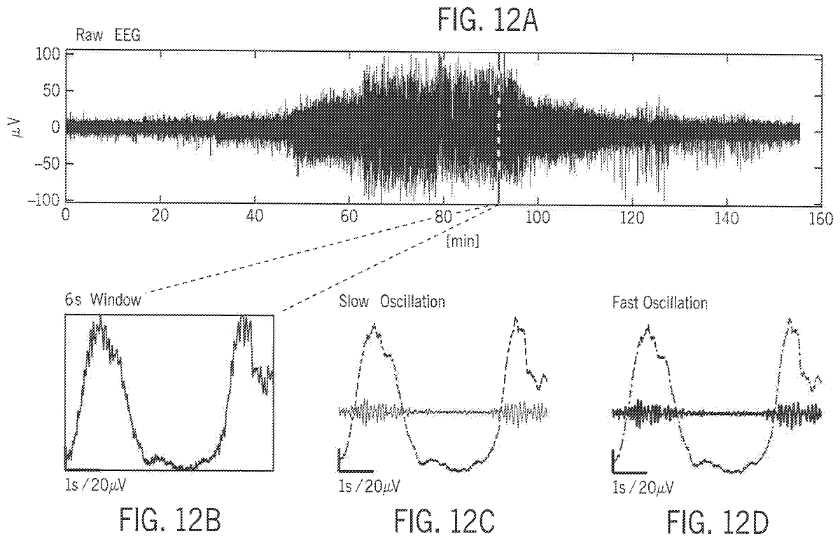

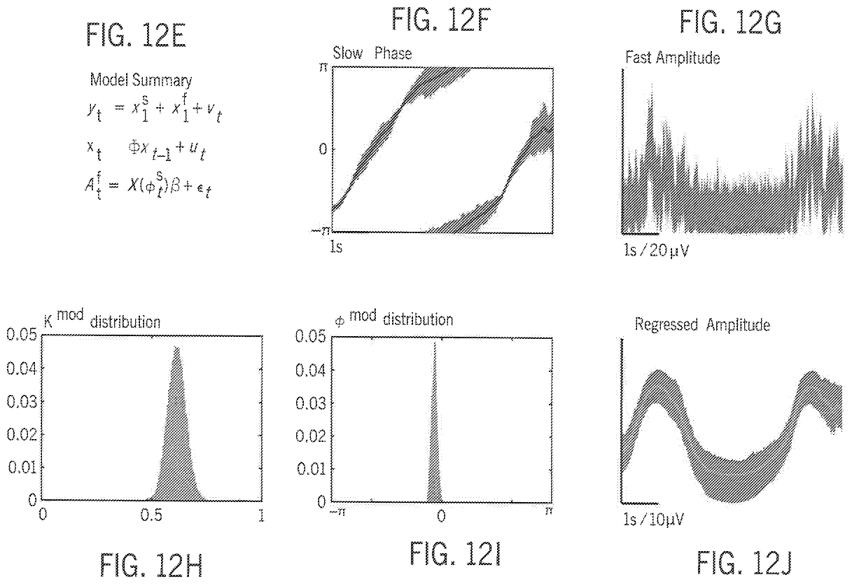

xₗⁱ=aⱼ [symbol] (ωⱼ)xₜ₋₁^{j}+uₖ^{j}˜ [symbol] (0,σⱼ²I_{2×2}) (2)

where aⱼ is a scaling parameter, [symbol] (ωⱼ) is a 2-dimensional rotation of angle ωⱼ (the radial frequency and σⱼ² the process noise variance. An example of this state space oscillation decomposition is shown in FIGS. 12A-D. This approach eliminates the need for traditional bandpass filtering since the slow and fast components are directly estimated under the model. Perhaps more importantly, the oscillations' respective components can be regarded as the real and imaginary terms of a phasor or analytic signal. As a result, a Hilbert transform, which is used in prior methods for synthesizing the imaginary signal component from the observed real signal, is no longer needed. Thus, the latent vector's polar coordinates provide a direct representation of the slow instantaneous phase of and fast oscillation amplitude Aₜ^{f} (FIGS. 12F and 12G). Note that xₜ=[xₜ^{sT}xₜ^{fT}]^{T} and obtain a canonical state space representation:

yₜ=Mxₜ+vₜ, vₜ˜ [symbol] (0,R) (3)

xₜ=Φxₜ₋₁+uₜ, uₜ˜ [symbol] (0,Q) (4)

where Φ∈ [symbol] ^{4×4} is a block diagonal matrix composed of the rotations described earlier, Q the total process noise covariance, R the observation noise covariance and M∈ [symbol] ^{1×4} links the observation with the oscillation first (real) coordinate. Estimates (Φ, Q, R) can be made using an Expectation-Maximization (EM) procedure or using numerical procedures to maximize the log-likelihood or log-posterior density. To extend this model to add more independent oscillation components or to account for the harmonics of an oscillation (as one might encounter in a nonlinear system) a more general formulation is described and derived below.

A more specific derivation of the state space model embodied by equations (3) and (4) is described below. For a time series sampled at Fₛ, we consider a time window {yₜ}ₜ₋₁^{N}∈ [symbol] ^{N}, and we assume, in this section, that yₜ is the sum of observation noise and components from two latent states xₜ^{s} and xₜ^{f}∈ [symbol] ^{2N} which account fora slow and a fast component. For j=s, f and t=2 . . . N, each component follows the process equation:

$$\begin{matrix}{x}_{t}^{j} = {a}_{j} ( {ω}_{j} ) {x}_{t - 1}^{j} + {u}_{t}^{j} , {u}_{t}^{j} ∼ ( 0 , {Q}_{j} ) & ( 5 ) \\ ( {ω}_{j} ) = ( \begin{matrix}cos ( {ω}_{j} ) & - sin ( {ω}_{j} ) \\ sin ( {ω}_{j} ) & cos ( {ω}_{j} )\end{matrix} ) \\ and & ( 6 ) \\ {Q}_{j} = ( \begin{matrix}{σ}_{j}^{2} & 0 \\ 0 & {σ}_{j}^{2}\end{matrix} ) . & ( 7 )\end{matrix}$$

where aⱼ∈(0,1) and [symbol] (ωⱼ) is a rotation matrix with angle ωⱼ=2πfⱼ/Fₛ

As previously stated, the phase ϕₜⁱ and amplitude Aₜ^{j} of each oscillation are obtained using the latent vector polar coordinates:

$${ϕ}_{t}^{j} = {tan}^{- 1} ( \frac{{x}_{2 , t}^{j}}{{x}_{1 , t}^{j}} ) and {A}_{t}^{j} = \sqrt{{( {x}_{1 , t}^{j} )}^{2} + {( {x}_{2 , t}^{j} )}^{2}} \qquad ( 8 )$$

Each oscillation has a broad-band power spectral density (PSD) with a peak at frequency fj. The parametric expression for this PSD will be derived further below.

Setting M=[1 0 1 0], xₜ=[xₜ^{sT} xₜ^{fT}]^{T}, and Q and Φ to be block diagonal matrices whose blocks are Qᵢ and aⱼ [symbol] (ωⱼ), respectively, we find the canonical state space of equations (3) and (4).

Given the observed signal yₜ, we aim to estimate both the hidden oscillations xₜ and their generating parameters (ϕ, Q, R). We do so using an Expectation-Maximization (EM) algorithm with an E-step and an M-step, a general derivation of which will be described below. The hidden oscillations xₜ are estimated in the E-step of the EM algorithm using the Kalman filter and fixed-interval smoother, while the generating parameters are estimated in each iteration of the M-step.

It is noted that the AR(2) form can represent oscillations with different amplitudes, and different levels of resonance or bandwidth. In addition, the AR(2) spectrum has a “1/f” profile at frequencies above the oscillatory frequency. More generally, a number of alternative scenarios where one or both of these oscillations are absent are possible. For instance, in the awake resting state, frontal EEG channels would not be expected to have an alpha oscillation, nor a slow oscillation. In this instance, broad-band “1/f” dynamics might be present, and could be modeled using an AR(1) process. Alternatively, a combination of AR(1) and AR(2) dynamics could also be represented in this state space form.

As a shorthand, we will refer to the model described in equations (3) and (4) as an “AR(2+2)” model, since there are two AR(2) processes in this model. The AR(2+2) model can be fit to EEG data in order to identify an alpha oscillation and a slow oscillation. The combination of AR(1) and AR(2) processes can be referred to as an “AR(1+2)” model. We will also consider models with a single dynamic component represented by either AR(1) or AR(2) state dynamics. As noted above, these models are technically autoregressive moving average (ARMA) models due to the presence of observation noise in equation (4), but we will refer to the models according to their underlying state dynamics to emphasize the oscillatory structure of the model. One or more models such as the AR(2+2) can be fit to EEG data using an expectation maximization algorithm for state space models such as a Shumway and Stoffer expectation maximization algorithm. Initial AR parameters can be set by a user. For example, an initial slow oscillation peak frequency can be set to 1 Hz, an initial alpha oscillation peak frequency can be set to 10 Hz, and the poles for both oscillations can be set initially at a radius of 0.99 from the origin in the complex plane. The expectation maximization algorithm can utilize Kalman filter, fixed-interval smoother, and covariance smoothing algorithms to further refine estimated parameters. The expectation step and the maximization step of the EM algorithm can each be iterated through a predetermined number of times, such as 200 iterations, before estimated parameters such as AR parameters and noise covariances are output. The iterations may also be continued until some convergence criteria are met, including but not limited to convergence of the parameters being estimated, or convergence in the value of the log-likelihood or log-posterior density. After AR parameters and noise covariances have been estimated, the Akaike Information Criterion (AIC) for different models can be calculated to compare how well each model fits a given EEG. The AIC is defined as AIC=−2 log(L)+2p, where L is the value of the likelihood for the model and p is the number of parameters in the model.

During a certain test to determine viability of the AR(2+2) model in accurately determining slow oscillations and alpha oscillations, EEG was recorded during induction of propofol-induced unconsciousness in healthy volunteers between the ages of 18 to 36 years. The EEG signals were recorded at sampling frequency of 5000 Hz, down-sampled to 200 Hz. A single frontal channel of EEG was analyzed by comparing an awake, eyes-closed baseline state to a propofol-induced unconscious state. In the propofol-induced unconscious state, the frontal EEG contains large slow (0.1-1 Hz) and alpha (8-12 Hz) oscillations. In the baseline state, these oscillations are absent, although a slow drift may be observed. The AIC was used to select a best model from among the AR(1), AR(2), AR(1+2), and AR(2+2) models. To account for the state space formulation of each model, we included the AR parameters, state noise variances, and observation noise variance as parameters in the AIC, all of which were estimated by the expectation maximization (EM) algorithm. Other information such as Bayesian Information Criteria (BIC) can also be used in place of the AIC in order to select a best model.

A signal can be decomposed into frequency-dependent components using bandpass filters. We therefore compared the performance of the state space models with bandpass filtered signals. To represent the slow wave, we used an 100 order Hamming-windowed low pass FIR filter with a 1 Hz cutoff frequency. To represent the alpha wave, an order 100 Hamming-windowed bandpass FIR filter with a passband between 8 and 12 Hz was used. A reconstructed bandpass signal by summing the bandpassed slow and alpha components was the created.

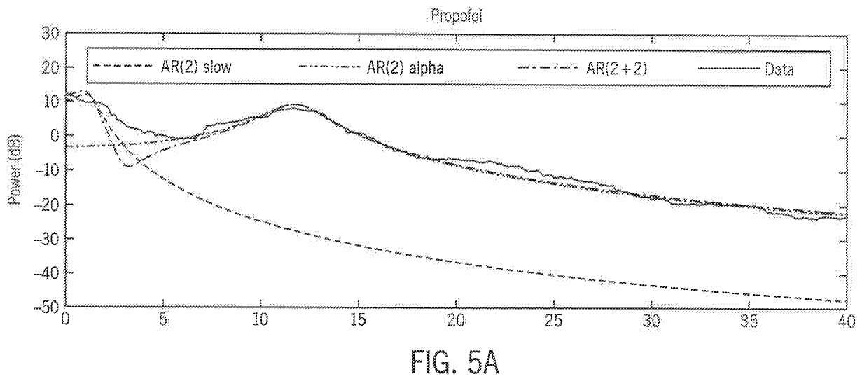

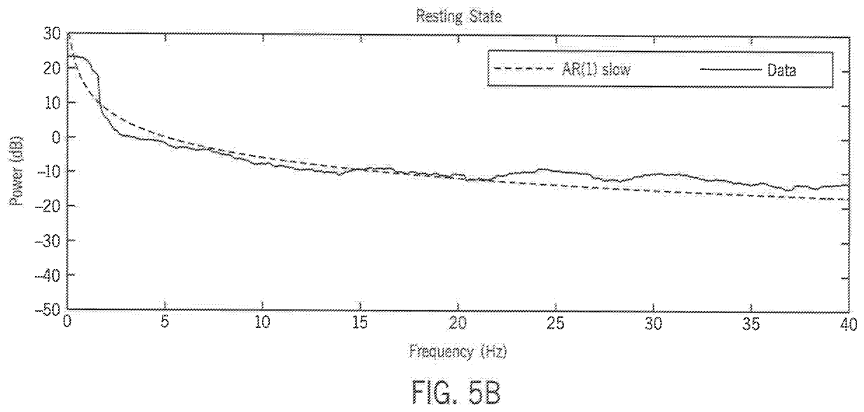

Table I below shows AIC scores for the AR(1), AR(2), AR(1+2), and AR(2+2) models for the propofol-induced unconscious condition. The AR(2+2) was shown to outperform the other models during the propofol-induced unconscious condition, as evidenced by having the lowest AIC level among all models. For the baseline case, the AIC is lowest for the AR(1) model, whereas for the propofol condition, the AIC is lowest for AR(2+2) model, allowing us to select these models for each respective condition. The spectra for the fitted models are shown in FIG. 5, alongside a nonparametric multitaper spectral estimate of the observed data (3 Hz spectral resolution, 26 tapers over 10 seconds of data). FIG. 5A shows the spectra for the AR(2+2) model in the propofol-induced case, while FIG. 5B shows the spectra for the AR(1) model in the resting state induced case.

TABLE I

| | Number of | | |
|---|---|---|---|
| Model | Parameters | Rest | Propofol |
| AR(1) | 3 | 6683.3 | 6664.6 |
| AR(2) | 4 | 6685.4 | 3728.8 |
| AR(1 + 2) | 6 | 6690.8 | 3567.8 |
| AR(2 + 2) | 7 | 6693.3 | 3407.4 |

There is no discernible alpha oscillation in the case of resting state, supporting the model selection of AR(1). The residuals between the modeled observed data and the collected data in resting state had a magnitude of 4.9348 uV or less. In this AR(1) case, there is only one hidden state that is nearly identical to the recorded data. The AR(1) model for this baseline condition is:

x_{slow,t}=0.9953x_{slow,t-1}+w_{slow,t}w_{slow}˜ [symbol] (0, 5.1593) (9)

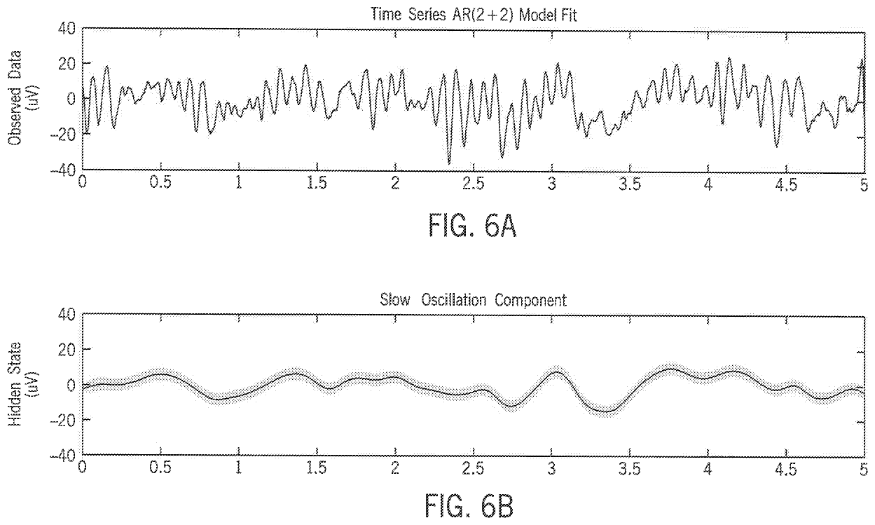

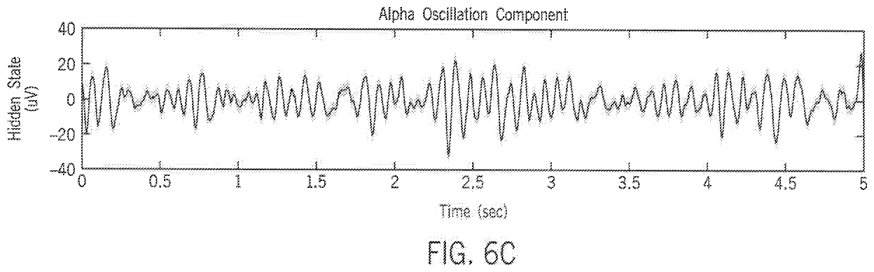

The spectrum of the AR(2+2) model representing the propofol-induced unconscious case is shown in FIG. 5A, again alongside a multitaper spectral estimate of the observed data. The slow and alpha oscillation components of the AR(2+2) model show a close correspondence to the peaks observed in the multitaper spectrum. The AR(2+2) model has independent hidden states representing the slow and alpha oscillations, making it possible to separate the oscillations in the time domain. FIG. 6 shows the slow, alpha and combined oscillation time series estimated under the AR(2+2) model. Specifically, FIG. 6A shows the combined oscillation time series, FIG. 6B shows the slow oscillation time series, and FIG. 6C shows the alpha oscillation time series. The residuals have a magnitude of 0.2198 uV or less.

In addition to the oscillatory form of the AR components of the state space model described in equation (5), (6), and (7), traditional AR model formulations may also be used. For instance, the AR models describing each component in this propofol case can be described as:

x_{slow,t}=1.961x_{slow,t-1}−0.9627x_{slow,t-2}+w_{slow,t}w_{slow}˜ [symbol] (0, 0.0062) (10)

x_{alpha,t}=1.7826x_{alpha,t-1}−0.9139x_{alpha,t-2}+w_{alpha,t}wₐₗₚₕₐ˜ [symbol] (0, 1.6368) (11)

Under this form of model, the characteristic features of the oscillations, such as their peak frequency, can be calculated directly from the model parameters. The peak frequency of each AR(2) component in radians is given by

$$ω = {cos}^{- 1} \frac{{a}_{1} ( {a}_{2} - 1 )}{4 {a}_{2}} \qquad ( 12 )$$

where in this context a₁ is the first time lag parameter and a₂ is the second. The peak frequency of the slow wave under propofol is 1.0118 Hz and the peak frequency of the alpha oscillation is 11.6928 Hz. The slow component during resting state has a peak frequency of 0 Hz by the definition of an AR(1) process, since an AR(1) process has a single real-valued pole at zero frequency. The poles of the AR model components determine the shape of the power spectrum and the peak frequencies. The radius of the pole determines the prominence of the peak frequency and in effect the damping of the oscillatory process. In this AR(2) model for slow wave oscillations under propofol, the radius of the poles is 0.99 and in the model for alpha, the radius is 0.956, showing that the slow wave is slightly more prominent, as expected.

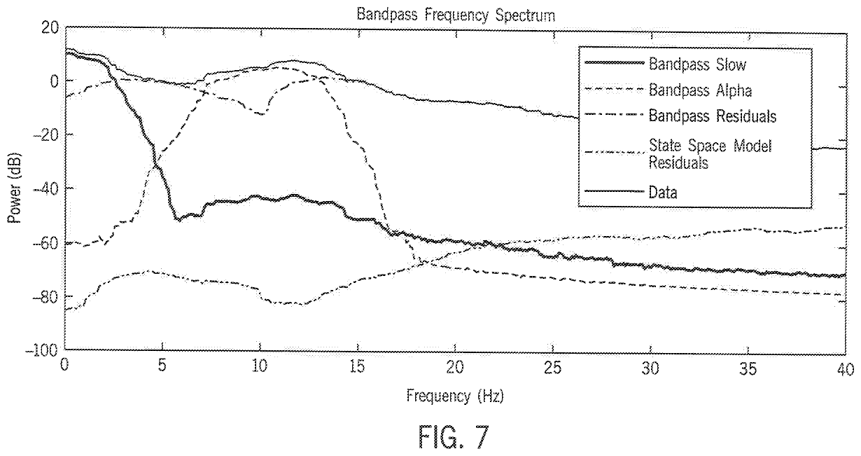

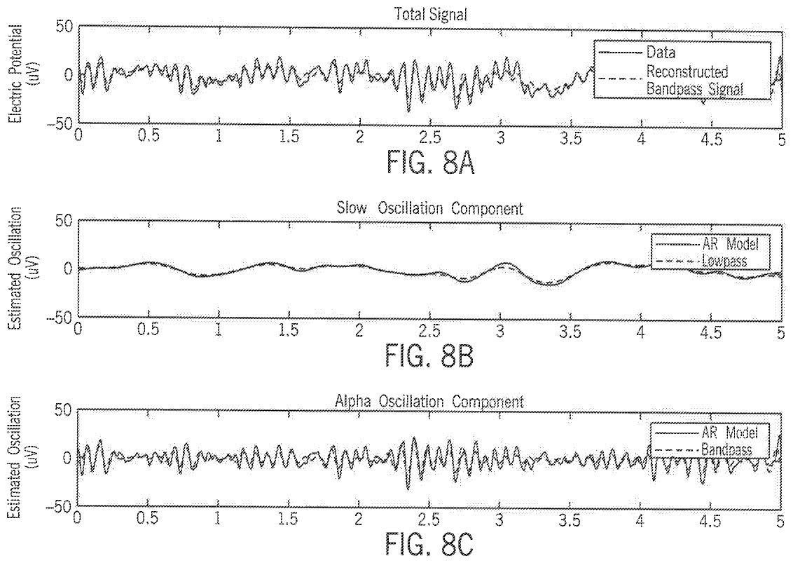

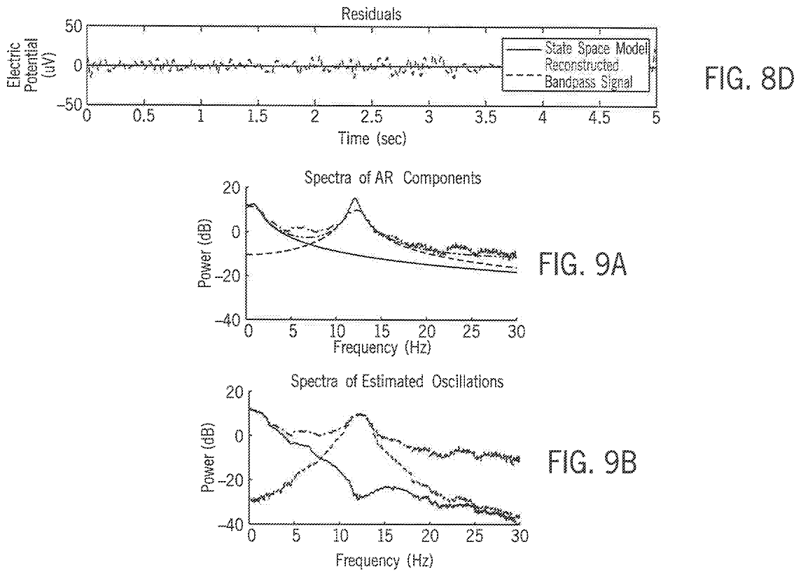

The performance of the state space model was compared with the more conventional bandpass filtered approach. FIG. 7 shows the multitaper spectra for the bandpass slow and alpha components, the reconstructed signal, and the residuals, alongside the residuals for the state space model. Although bandpass filtering identifies oscillations similar to those obtained using the state space model, the residuals under the bandpass method show prominent low frequency oscillatory structure. By comparison, the residuals from the state space model are approximately 60 dB smaller and less structured. In the bandpassed model, the power and structure in the residuals are a consequence of leakage outside of the arbitrary bandpass cutoffs, which our method avoids by using AR models to represent the component oscillations. FIG. 8 shows the component oscillations, reconstructed signals, and residuals for the bandpass method in time domain, alongside comparisons to the state space. FIG. 8A shows the total signal for the bandpass method and the state space method, FIG. 8B shows the slow oscilation component for the bandpass method and the state space method, FIG. 8C shows the alpha oscillation component for the bandpass method and the state space method, and FIG. 8D shows the residuals for the bandpass method and the state space method. Similar to the frequency domain analysis, we see significant time domain residual structure under bandpass filtering that is absent under the state space model. The state space model can therefore be used to identify oscillations without certain drawbacks such as relatively large residuals of bandpass-based methods.

Our results illustrate how state space models, structured in a particular form consisting of a combination of first and second-order systems, can be used to identify oscillatory structure in neural time series. A typical approach taken in neuroscience analyses is to compute the power within a spectral band, or variance in a bandpass filtered signal. A major limitation of this approach is that broadband noise spanning the frequency range of interest cannot be distinguished from an oscillation. Our approach addresses this limitation by explicitly modeling potential oscillatory components, and then selecting amongst a set of models with different oscillatory configurations (i.e. AR(1), AR(2), AR(1+2), and AR(2+2)) to determine the composition of the signal. Under this approach, power attributed to a specific oscillatory component can be calculated, independent of other broad-band components, and the component oscillatory time series can be separated.

This state space approach differs from traditional AR or ARMA modeling by specifying a structure for the AR parameters to represent either drift (AR(1) or AR(2) with real poles) or oscillatory terms (AR(2) with complex poles). Although a more general AR or ARMA model can certainly identify such components, our experience is that the spectral representations of such models can be highly variable as the model order increases. By positing a specific form for the putative drift terms or oscillations, we can reduce the number of model parameters required to represent such features, and increase the robustness of the model estimates. Moreover, the structure of our proposed models is more easily interpreted in terms of specific oscillatory components than a more general AR or ARMA structure.

The state space method has an advantage over certain methods in that oscillatory components can be separated in the time domain. The linear systems approach we have described may also provide more specificity in detecting oscillations, since the temporal structure or impulse response implied by the AR components is specific to oscillators. Furthermore, the state space method can be used on unprocessed (i.e. not bandpass filtered) EEG data, thereby avoiding some disadvantages of bandpass filtering.

Prior Distributions for the Model Parameters. When fitting certain EEG data with models, a model may not fit accurately some of the observed oscillations if the observed oscillations have low power or are indistinguishable from the broader frequency spectrum landscape. With previously described methods, the only control that a user has over the model is in choosing the initialization parameters. A component intended to represent an oscillation with lower power can, during the fitting procedure, shift into a frequency range described by another oscillatory component, particularly if that component is large and broad-band. When evaluated using traditional model selection procedures such as the Akaike Information Criterion (AIC), these overfitted models can have lower AIC values. In part, this is because AIC tends to favor state space models with more components. The state space model described above may be sensitive to initialization and may require additional constraints based on prior knowledge of the neural oscillations being studied.

As a first exemplary case, EEG data with a strong alpha oscillation is fit using an AR(2+2) state space model. The spectra of AR components of the EEG data is shown in FIG. 9A, while the spectra of the estimated oscillations is shown in FIG. 9B. The state space model tends to fit well for the EEG data, which has a strong alpha oscillation.

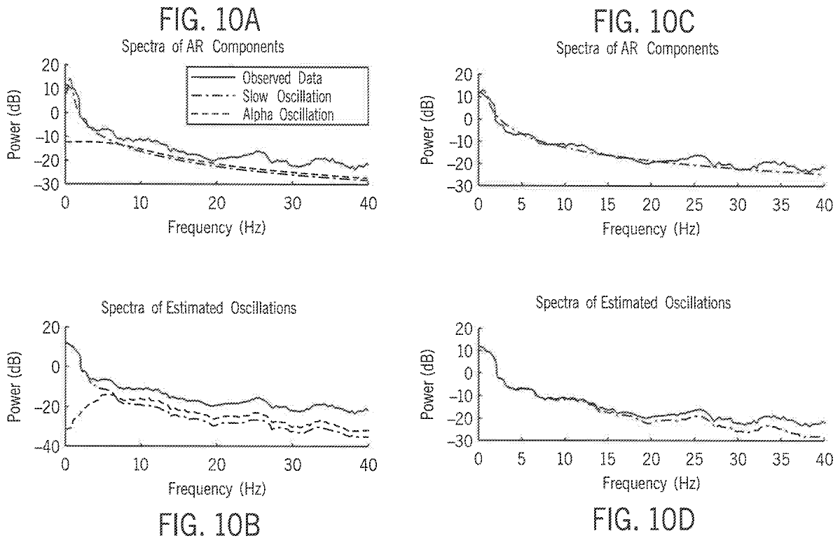

In contrast, a model may be unsuccessfully fit when an alpha oscillation is absent. In the case that an alpha oscillation is absent, the alpha component is incorrectly fit to the slow/delta frequency range. With no oscillatory power to anchor the alpha (8-12 Hz) component of the model to the intended frequency range, during the model fitting procedure this component shifts down in frequency towards the larger slow wave component. The AIC is lower for this model than the model with just a slow component, and would suggest that the “alpha+slow” model (AR(2+2)) should be selected. However, this goes against our intuition, since there is no apparent alpha oscillation in the (nonparametric) spectral estimate. Accordingly, the alpha component no longer resides in the alpha frequency rating, but instead as migrated into the slow wave frequency range, with a peak at 1.0872 Hz. The spectra of AR components for a slow and alpha model without a prior distribution on the center frequency, which will be explained below, is shown in FIG. 10A, while the spectra of estimated oscillations for the slow and alpha model with the prior distribution is shown in FIG. 10B. The spectra of AR components for a slow without alpha model is shown in FIG. 10C, while the spectra of estimated oscillations for the slow without alpha model is shown in FIG. 10D.

To make this class of models more identifiable and more robust to inappropriate model fitting, prior distributions can be used for the oscillatory frequency and radius or damping factor, respectively.

For a prior probability distribution on the center frequency of a single component, the model may need to have access to a range of potential frequencies, yet remain confined approximately to a pre-defined frequency range, i.e., to anchor the component to a specific frequency band.

We selected a Von Mises distribution to serve as the prior probability distribution. The Von Mises distribution is a symmetric probability distribution with a domain spanning 0 Hz to the sampling frequency. It takes the form:

$$p ( ω \vert μ , κ ) = \frac{1}{2 π {I}_{0} ( κ )} {e}^{κ cos ( ω - μ )} \qquad ( 13 )$$

where ω represents the center frequency of the oscillation that we wish to constrain with the prior distribution, I₀ is a modified Bessel function of order 0, and κ and μ are hyperparameters describing the shape of the distribution. The parameter μ sets the mean or center frequency for the prior distribution. The concentration parameter K determines the width or bandwidth of the distribution and determines the strength of the anchor to the intended frequency band. Other probability distributions with similar properties could also be considered for this purpose of anchoring an oscillatory AR component to a specific frequency range. The Von Mises prior can make model fitting more robust, since frequency components are constrained to a limited frequency range, and thus cannot overlap with other oscillatory components during the model fitting or parameter estimation process. The test case below shows where the model without the Von Mises prior breaks down: without the Von Mises Prior AIC selects the two component model for this subject when in fact there is only one oscillation in the slow band. Notably, the selected model fits two oscillations in the slow band: In the absence of the Von Mises prior, the higher frequency alpha component drifts into the slow band, centered at 1.0782 Hz.

The Von Mises Prior parameters controlling center frequency and concentration can be selected based on the frequency band desired. These can be, for instance, determined based on the width and center of the generally accepted frequency bands in neuroscience literature. For example, the slow and alpha model in Table II below had a Von Mises prior on the alpha frequency component using a center frequency of 10 Hz and a width of ˜1.2 Hz at a probability value of 0.367 to help anchor the alpha component to the alpha frequency range.

By adding the Von Mises prior, the component stays in the desired range, and given the lack of alpha power in the data, the component has little power assigned to it and has a broad bandwidth. When AIC is applied, it has a higher value than the model with just a slow oscillation (and no alpha). Thus, among models using the Von Mises prior, AIC selects the correct model. This illustrates how use of the Von Mises prior increases model robustness, allowing model selection criteria such as AIC to choose the correct model. As we will show below, this increased robustness also enables algorithms for iteratively fitting additional oscillatory components. The AIC, slow frequency, slow radius (damping factor), alpha frequency, and alpha radius (damping factor) for the slow and alpha without prior model, the slow and no alpha model, and slow and alpha with prior model are shown in Table II.

TABLE II

| | Slow and Alpha, | Slow and No | Slow and Alpha, |
|---|---|---|---|
| Model | No prior | Alpha | with prior |
| AIC | 6990.5 | 7003.9 | 7009.4 |
| Slow | 1.800 Hz | 1.4008 Hz | 1.5036 Hz |
| Frequency | | | |
| Slow Radius | 0.9618 | 0.9644 | 0.9649 |
| (damping | | | |
| factor) | | | |
| Alpha | 1.0782 Hz | N/A | 9.9951 Hz |
| Frequency | | | |
| Alpha Radius | 0.8120 | N/A | 0.3338 |
| (damping | | | |
| factor) | | | |

By a similar rationale, other parameters may be sensitive to initialization and adding a prior distribution on these parameters may increase robustness of the model fitting and model selection, enabling more complete automation in model selection.

One such parameter is the radius or damping factor. The radius or damping factor controls the stability of the individual components and thus the stability of the entire system. As the radius or damping factor increases beyond 1, the oscillation time series will become unstable, and the amplitude of the time series will grow towards infinity. To keep the system stable, we can control the damping factor with a beta distribution to keep the parameter between 0 and 1. The beta distribution's shape is controlled by two parameters and has a domain from 0 to 1, and is described by the below equation:

$$ρ ( a \vert α , β ) = \frac{Γ ( α + β )}{Γ ( α ) Γ ( β )} {{a}^{α - 1} ( 1 - a )}^{β - 1} \qquad ( 14 )$$

Where a is the radius or damping factor, and α and β are hyperparameters that determine the shape of the prior distribution.

As described above, a model selection procedure commonly used to fit AR processes is the Akaike Information Criterion (AIC). The model fitting steps and selection procedure are diagrammed below. The iteration of the maximization step and the expectation step comprises the expectation maximization algorithm. The Kalman Filter and Fixed Interval Smoother are necessary to estimate the time series of the hidden states, also referred to as the estimated oscillation time series. Expectation-Maximization (EM) Algorithm for Independent and Harmonic Oscillation Decompositions

An exemplary expectation maximization EM algorithm capable of estimating independent and harmonic oscillations is now described. The EM algorithm can be used during model fitting to iteratively search for model parameters that allow for a best fit for a given model. The model includes two main steps: maximization and expectation. The expectation and maximization steps can be performed a predetermined number of times, such as two hundred times, in order to reach an optimal solution. The EM algorithm can accept a model with initialized AR parameters and output estimated AR parameters for the model.

Since all noise terms are assumed to be additive Gaussian, the complete data log likelihood for one time window of length N is:

$$log L = log p ( {x}_{1} , … , {x}_{N} , {y}_{1} , … , {y}_{N} ) {=}^{•} - \frac{N}{2} log \vert Q \vert - \frac{1}{2} {\sum }_{t = 1}^{N} {( {x}_{t} - Φ {x}_{t - 1} )}^{T} {Q}^{- 1} ( {x}_{t} - Φ {x}_{t - 1} ) - \frac{N}{2} log \vert R \vert - \frac{1}{2} {\sum }_{t = 1}^{N} {( {y}_{t} - {Mx}_{t} )}^{T} {R}^{- 1} ( {x}_{t} - {Mx}_{t} ) \qquad ( 15 )$$

We wish to maximize log L with respect to Θ=(Φ, Q, R) but we do not have access to the hidden oscillations xₜ. We use the EM algorithm to alternatively and iteratively estimate (at the E-Step) and maximize (at the M-Step) the log likelihood. At iteration r, we use the Kalman filter to estimate xₜ given a set Θᵣ which gives us access to:

Gᵣ(Θ)= [symbol] ᵣ(log L|{yₜ}ₜ₌₁^{N})

Then, we deduce Θᵣ₊₁:

$${Θ}_{r + 1} = {argmax}_{Θ} {G}_{r} ( Θ ) \qquad ( 17 )$$

The Kalman filter can be used to estimate the hidden oscillations given the observations and the model parameters. First, the state at the next time point is predicted and is then compared to the observation. An updated estimate based on the most recently observed data can then be produced. Given the full observation time series, we can apply backward smoothing to refine the update to account for the full observation series (i.e., fixed interval smoothing).

For t, t₁, t₂=1 . . . N, we note:

xₜ^{N}= [symbol] ᵣ(xₜ|{yₜ}ₜ₌₁^{N}), Pₜ_{₁}_{,t}_{₂}^{N}=covᵣ(xₜ_{₁}, xₜ_{₂}|{yₜ}ₜ₌₁^{N}), and Pₜ^{N}=P_{t,t}^{N} (18)

and compute those quantities using the forward smoothing algorithm as follows:

Prediction: xₜ^{t-1}=Φxₜ₋₁ⁱ⁻¹

Pₜ^{l-1}=ΦPₜ₋₁^{t-1}Φ^{T}+Q

Kalman Gain: Kₜ=Pₜⁱ⁻¹M^{T}(MPₜ^{t-1}M^{T}+R)⁻¹

Update: xₜⁱ=xₜ^{t-1}+Kₜ(yₜ−Mxₜ^{t-1})

Pₜ^{t}=Pₜ^{t-1}−KₜMPₜ^{t-1} (19)

and a set of backwards recursions. For t=N . . . 1 and t₁<t₂:

Backward Gain: Jₜ₋₁=Pₜ₋₁^{t-1}Φ^{T}(Pₜ^{t-1})⁻¹

Smoothing: xₜ₋₁^{N}=xₜ₋₁^{t-1}+Jₜ₋₁(xₜ^{N}−Φxₜ₋₁^{t-1})

Pₜ₋₁^{N}=Pₜ₋₁^{t-1}+Jₜ₋₁(Pₜ^{N}−Pₜ^{t-1})Jₜ₋₁^{T}

Covariance: Pₜ_{₁}_{,t}_{₂}^{N}=Jₜ_{₁}Pₜ_{₁}_{+1,t}_{₂}^{N} (b 20)

The E-step of the EM algorithm is now described. The Kalman filter estimation methods described above may be included in the E-step of the EM algorithm. Here we describe Gᵣ(Θ) for state space oscillation decomposition with harmonic components. A single oscillation is defined by a rotation matrix [symbol] (ω), a scaling parameter a and a process noise covariance matrix Q=σ²I_{2×2}. In what follows, we consider d independent oscillations, which are respectively associated to h₁, . . . , h_{d} harmonics. For j=1 . . . d, an oscillation with fundamental frequency ωⱼ is the sum of h=1 . . . hⱼ, harmonics respectively defined by [symbol] (hωⱼ), a_{j,h} and Q_{j,h}−σ_{j,h}²I_{2×2} We note D=Σⱼ₌₁^{d}hⱼ the total number of oscillatory components.

For V∈ [symbol] ^{2D×2D}, j=1 . . . d and h=1 . . . hⱼ, we note V_{j,h} the 2 by 2 diagonal block associated with the hⱼ^{th} harmonic of oscillation j. Φ and Q are block diagonal matrices whose diagonal blocks are a_{j,h} [symbol] (h_{j,h}) and Q_{j,h}:

Φ=diag(a_{1,1} [symbol] (ω₁), . . . a_{1,h}_{₁} [symbol] (h₁ω₁), . . . , a_{d,1} [symbol] (ω_{d}), . . . a_{d,h} [symbol] (h_{d}ω_{d})) (21)

Q=diag(Q_{1,1}, . . . Q_{1,h}_{₁}, . . . , Q_{d,1}, . . . Q_{d,h}_{_{d}}) (22)

Additionally, we will use M=[1 0 1 0 . . . 0]∈ [symbol] ^{2D} and for U∈note: rt(U):=U₂₁−U₁₂ and tr(U)=U₁₁+U₂₂. Taking the conditional expectation of the log likelihood log L at iteration r for a fixed set of parameter Θᵣ=(Φ, Q, R), we obtain:

$$\begin{matrix}{G ( Φ , Q , R )}_{r} \mathring{=} - \frac{N}{2} log \vert Q \vert - \frac{1}{2} tr ( {Q}^{- 1} ( C - B {Φ}^{T} - Φ {B}^{T} + Φ A {Φ}^{T} ) ) - \frac{N}{2} log \vert R \vert - \frac{1}{2} tr ( {R}^{- 1} ( {\sum }_{t = 1}^{N} ( {y}_{t} - {Mx}_{t}^{N} ) {( {y}_{t} - {Mx}_{t}^{N} )}^{r} + {MP}_{t}^{N} {M}^{T} ) \mathring{=} {G ( Φ , Q )}_{r} + {C ( R )}_{r} \\ where : & ( 23 ) \\ A = {\sum }_{t = 1}^{N} ( {P}_{t - 1}^{N} + {{x}_{t - 1}^{N} ( {x}_{t - 1}^{N} )}^{T} ) \\ B = {\sum }_{t = 1}^{N} ( {P}_{t , t - 1}^{N} + {{x}_{t}^{N} ( {x}_{t - 1}^{N} )}^{T} ) \\ C = {\sum }_{t = 1}^{N} ( {P}_{t}^{N} + {{x}_{t}^{N} ( {x}_{t}^{N} )}^{T} ) & ( 24 )\end{matrix}$$

At the M-Step, Gᵣ can be maximized with respect to R and (Φ, Q) independently. We have:

$$\frac{\partial {G}_{r}}{\partial R} ( {R}_{r + 1} ) = 0 ⟺ {R}_{r + 1} = \frac{1}{N} {\sum }_{t = 1}^{N} ( {( {y}_{t} - {Mx}_{t}^{N} )}^{2} + {MP}_{t}^{N} {M}^{T} ) \qquad ( 25 )$$

Since Q is block) diagonal, we can write:

$$tr ( {Q}^{- 1} V ) = {\sum }_{j = 1}^{d} {\sum }_{h = 1}^{{h}_{j}} tr ( {Q}_{j , h}^{- 1} {V}_{j , h} ) = {\sum }_{j = 1}^{d} {\sum }_{h = 1}^{{h}_{j}} \frac{1}{{σ}_{j , h}^{2}} tr ( {V}_{j , h} ) \qquad ( 26 )$$

A is symmetric and (I) is a block diagonal matrix whose element are 2×2 rotation matrices, we develop equation (23) and obtain:

$${G}_{r} ( Φ , Q ) {=}^{a} - N {\sum }_{j = 1}^{d} {\sum }_{h = 1}^{{h}_{j}} log {σ}_{j , h}^{2} - {\sum }_{j = 1}^{d} {\sum }_{h = 1}^{{h}_{j}} \frac{1}{2 {σ}_{j , h}^{2}} [ tr ( {C}_{j , h} ) - 2 {a}_{j , h} ( tr ( {B}_{j , h} ) cos ( h {ω}_{j} ) + rt ( {B}_{j , h} ) sin ( h {ω}_{j , h} ) ) + {a}_{j , h}^{2} tr ( {A}_{j , h} ) ] \qquad ( 27 )$$

Differentiating with respect to process noises covariances σ_{j,h}², scaling parameter a_{j,h} and fundamental frequencies ωⱼ yields:

$$( 28 )$$

$$\{ \begin{matrix}\frac{\partial G}{\partial {a}_{j , h}} ( Φ , Q ) = 0 & ⟺ & {a}_{j , h} tr ( {A}_{j , h} ) = tr ( {B}_{j , h} ) cos ( h {ω}_{j} ) + rt ( {B}_{j , h} ) sin ( h {ω}_{j} ) \\ \frac{\partial G}{\partial {σ}_{j , h}^{2}} ( Φ , Q ) = 0 & ⟺ & {a}_{j , h}^{2} = \frac{1}{2 N} ( tr ( {C}_{j , h} ) - {a}_{j , h}^{2} tr ( {A}_{j , h} ) ) \\ \frac{\partial G}{\partial {ω}_{j}} ( Φ , Q ) = 0 & ⟺ & \begin{matrix}{\sum }_{h = 1}^{{h}_{j}} \frac{h}{{σ}_{j , h}^{2}} [ {a}_{j , h} ( tr ( {B}_{j , h} ) sin ( h {ω}_{j} ) - \\ rt ( {B}_{j , h} ) cos ( h {ω}_{j} ) ) ] = 0\end{matrix}\end{matrix}$$

We substitute the upper equations of (28) into the bottom (i.e. third) equation and we note:

$${\tilde{ω}}_{j , h} = \frac{1}{h} {tan}^{- 1} ( \frac{rt ( {B}_{j , h} )}{tr ( {B}_{j , h}} ) and {S}_{jh} = \frac{2 tr ( {A}_{j , h} ) tr ( {C}_{j , h} )}{{rt ( {B}_{j , h} )}^{2} + {tr ( {B}_{j , h} )}^{2}} - 1 \qquad ( 29 )$$

Using trigonometric identities, equations (28) can finally be rewritten:

$$( 30 )$$

$$\{ \begin{matrix}\frac{\partial G}{\partial {a}_{j , h}} ( Φ , Q ) = 0 & ⟺ & {a}_{j , h} tr ( {A}_{j , h} ) = tr ( {B}_{j , h} ) cos ( h {ω}_{j} ) + rt ( {B}_{j , h} ) sin ( h {ω}_{j} ) \\ \frac{\partial G}{\partial {a}_{j , h}^{2}} ( Φ , Q ) = 0 & ⟺ & {a}_{j , h}^{2} = \frac{1}{2 N} ( tr ( {C}_{j , h} ) - {a}_{j , h}^{2} tr ( {A}_{j , h} ) ) \\ \frac{\partial G}{\partial {a}_{j , h}} ( Φ , Q ) = 0 & ⟺ & {\sum }_{h = 1}^{{h}_{j}} \frac{h sin ( 2 h ( {ω}_{j} - {\tilde{ω}}_{j , h} ) )}{{S}_{j , h} - cos ( 2 h ( {ω}_{j} - {\tilde{ω}}_{j , h} ) )} = 0\end{matrix}$$

Overall, for j=1 . . . d, if hⱼ>1, we numerically solve for ωⱼ using equation (30) and deduce a_{j,h} and σ_{j,h}² for h=1 .. . hⱼ.

If hⱼ=1, we immediately have:

$${w}_{j} = {tan}^{- 1} ( rt ( {B}_{j} ) \text{/} tr ( {B}_{j} ) ) \\ {a}_{j} = \frac{\sqrt{{rt ( {B}_{j} )}^{2} + {tr ( {B}_{j} )}^{2}}}{tr ( {A}_{j} )} \\ {σ}_{j}^{2} = \frac{1}{2 N} ( tr ( {C}_{j} ) - {a}_{j}^{2} tr ( {A}_{j} ) ) \qquad ( 31 )$$

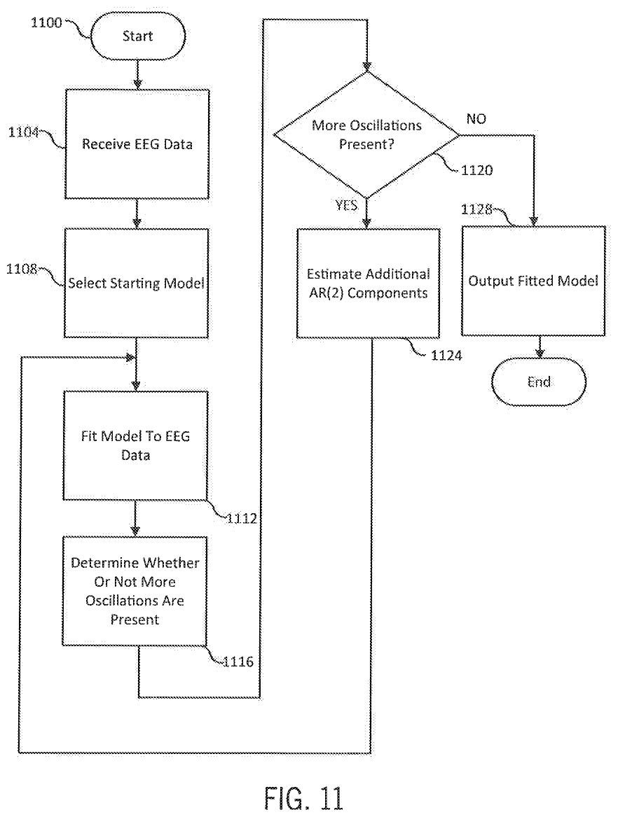

Referring to FIG. 11, an exemplary process 1100 for fitting EEG data with a state space model is shown. The process 1100 can include using a prior distribution to fit alpha oscillations and using an EM algorithm capable of detecting harmonics as described above. The state space model can include several oscillation components and harmonic oscillations of any of those components, depending on the EEG data (i.e. if a patient is resting, awake, unconscious, etc.). The state space model for the propofol example case can include a slow wave, an alpha oscillation, and harmonic oscillations of the alpha oscillation. The state space model can also include additional non-harmonic oscillations depending on the EEG data. For example, the process 1100 may fit a slow oscillation component and an alpha oscillation with or without harmonic oscillations if a patient has been administered propofol. The process 1100 can be implemented as instructions on at least one memory. The instructions can then be executed by at least one processor.

At 1104, the process 1100 can receive EEG data. The EEG data can be received from a patient monitoring device such as patient monitoring device 202 described above in conjunction with FIG. 2. The EEG data can be interpreted as a waveform. The process can then proceed to 1108.

At 1108, the process can select a starting model. In some embodiments, the process 1100 can select a starting model by fitting an AR model of large order, identifying a target order with the lowest AIC, and then identifying the most prominent frequency peak(s) of the target order to determine the starting parameters of the AR(2) components. The starting parameters for the propofol example case can include initial poles for each AR(2) component (i.e oscillation), which define the initial slow oscillation peak frequency and the initial peak alpha oscillation peak frequency. The initial pole includes information that can be used to determine a peak frequency. In some embodiments, the process 1100 can select a model with the lowest number of components, such as only a single AR(2) component representing a slow oscillation. In later steps, the process 1100 can determine if there are more components and add additional components to the model. Any of the AR(2) components can have one or more prior distributions on model parameters. The prior distributions may allow the model to be more accurately fit to a given oscillation. As described above, AR(2) components in the alpha oscillation range can have a Von Mises prior on the oscillation frequency in order to better fit certain alpha oscillation, such as relatively weak alpha oscillations. The Von Mises prior can have a center frequency of 10 Hz and a width of about 1.2-2 Hz at a probability value of 0.367. As described above, a beta distribution can be used to represent the prior distribution for the damping factor of the model. The beta distribution can keep the damping factor between 0 and 1 by having a domain from 0 to 1. The model can then proceed to 1112.

At 1112, the process 1100 can fit the model to the EEG data. The process 1100 can isolate line noise by fixing the noise frequencies, which may occur above the alpha frequency range. The process 1100 can fit the model using an EM algorithm. The EM algorithm can be the EM algorithm described in the “Expectation-Maximization (EM) Algorithm for Independent and Harmonic Oscillation Decompositions” section described above. The EM algorithm can fit any number of independent oscillations, each of which are associated with one or more harmonics. In this way, the presence of harmonics of the independent oscillations (i.e. a slow wave and an alpha oscillation) can be detected even if there are other independent oscillations near the harmonics. As described above, an expectation step and EM algorithm may iterate through an expectation step and a maximization step of the EM algorithm can each be iterated through a predetermined number of times, such as 200 iterations, before estimated parameters such as AR parameters and noise covariances are output. The iterations may also be continued until some convergence criteria are met, including but not limited to convergence of the parameters being estimated, or convergence in the value of the log-likelihood or log-posterior density. The final estimated parameters such as oscillator frequency can then be used by a practitioner or another process to analyze the oscillators. After the EM algorithm has iterated through the expectation step and maximization step the predetermined number of times, the process 1100 can proceed to 1116.

At 1116, the process 1100 can determine whether or not more oscillations are present. In some embodiments, the process 1100 can determine if the EEG data has residuals or innovations that cannot be explained by white noise alone. The process 1100 can subtract the predicted estimated time series (i.e. xₜ^{t-1} from the Kalman filter above) from a time series of the EEG data to obtain a time series of the innovations. The process 1100 can determine if significant peaks exist in the frequency spectrum of the innovations. White noise generally has a constant frequency spectrum. The process 1100 can calculate a cumulative power magnitude plot of the time series of the innovations and determine if the cumulative power magnitude plot differs statistically significantly from a constantly increasing power plot. If no statistically significant difference is found between the innovations and white noise, the process 1100 can determine that no more oscillations are present in the EEG data, and that the model fitted in 1116, which can be referred to as a fitted model, should be used to approximate the oscillations of the EEG data. If one or more statistically significant differences are present, the process can determine that more oscillations are present in the EEG data. The process 1100 can determine whether or not the cumulative power magnitude of the innovations in constantly increasing, as is consistent with white noise, to determine whether or not the innovations are consistent with white noise. In some embodiments, the process 1100 can simply calculate an AIC score for the model and compare the AIC score to an AIC score of a previous model previously fitted to the data (i.e. at a prior execution of 1116). In later steps, the process 1100 will add more oscillation components to the model in order to better fit the model if more oscillations are present. If AIC scores of models keep improving with additional oscillation components, the process 1100 can keep adding oscillations components until AIC scores worsen. If the AIC score of the current model is better than the previous model, the process can determine that more oscillation(s) are present in the EEG data. If the AIC score of the current model is worse than the previous model, the process can determine more oscillation(s) are not present in the EEG data and that the previous model should be used as the fitted model to estimate oscillations in the EEG data. The process can then proceed to 1120.

At 1120, if the process 1100 determined that no more oscillations are present at 1116 (“NO” at 1120), the process can proceed to 1128. If the process determined that more oscillations are present at 1116 (“YES” at 1120), the process can proceed to 1124.

At 1124, the process 1100 can estimate an additional AR(2) component and associated parameters. An AR model of a large order can be fit to the time series of the innovations, and a pair of complex poles can be selected that contribute the most to a tallest peak of the AR component. Initial parameters can then be determined based on the pair of complex poles. Alternatively, a center frequency of the additional AR(2) component can be estimated from the time series of the residuals by estimating power of the time series and estimating peak frequency based on the estimated power. Alternatively, the center frequency can be estimated based on the power spectrum of the innovations. A damping factor of the additional AR(2) component can be initialized as an average from previous data sets or identified from the shape of the cumulative power magnitude plot. The estimated center frequency and damping factor can provide the same information provided from the pair of complex poles. The time series can be filtered with a lowpass filter before the center frequency and/or damping factor are derived. Alternatively, the process can estimate the center frequency and damping parameter based on the residuals using the FOOOF algorithm described by Halleret al. The process can then proceed to 1112.

At 1128, the process 1100 can output the fitted model including estimated AR parameters and noise parameters. The process 1100 can output the fitted model to a display for viewing by a user and/or to a memory for storage and/or use by another process. The fitted model can be the last model fitted 1116 before it was determined no more oscillation were present in 1120. The fitted model can be a model with the best AIC score if the process 1100 iteratively adds more components to the model until AIC scores worsen. The process 1100 may generate a report based on the fitted model and output the report to make the fitted model more easily viewable to a human. The process 1100 can then end.

The state space model and associated fitting methods described above can be used to perform cross-frequency analysis on EEG data. Cross-frequency analysis includes any analysis of relationships between oscillations, for example, between the phase or amplitude of a first oscillation and the phase or amplitude of a higher frequency oscillation. Phase-amplitude coupling (PAC) is a common example, where the phase of a first oscillation modulates the amplitude of a second oscillation. PAC can be performed across a range of frequencies, or between the phase of an oscillation and the amplitude of a broadband (non-oscillatory) signal. We also present a new method to estimate the relationship between the real and imaginary part of a first oscillation (represented as an analytic signal, as described above) and the amplitude of a second oscillation, range of frequencies, or broadband signal.

The standard approach for PAC analysis uses binned histograms to quantify the relationship between phase and amplitude, which is a major source of statistical inefficiency. The raw signal yₜ is first bandpass filtered to isolate slow and fast oscillations. A Hilbert transform is then applied to estimate the instantaneous phase of the slow oscillation ϕₜ^{s}, and instantaneous amplitude of the fast oscillation A. At time t, the alpha amplitude Aₜ^{f} is assigned to one of (usually 18) equally spaced phase bins of length δψ based on the instantaneous value of the slow oscillation phase: ϕₜ^{s}. The histogram is constructed over some time window T of observations, for instance a ˜2 minute epoch, which yields the phase amplitude modulogram (PAM):

$$P A M ( T , ψ ) = \frac{{\int }_{- δ t \text{/} 2}^{δ t \text{/} 2} {\int }_{ψ - δφ \text{/} 2}^{ψ + δφ \text{/} 2} {A}_{t}^{f} δ ( {ϕ}_{t}^{s} - {ψ}^{′} ) dtd {ψ}^{′}}{2 π {\int }_{t - δ t \text{/} 2}^{t + δ t \text{/} 2} {A}_{t}^{f} dt} \qquad ( 32 )$$

For a given window T, PAM(T, .) is a probability distribution function which assesses how the fast oscillation amplitude is distributed with respect to the slow oscillation phase. The strength of the modulation is then usually measured with the Kullback-Leibler divergence with a uniform distribution. It yields the Modulation Index (MI):

MI(T)=∫_{-π}^{π}PAM(T, ψ)log₂[2πPAM(t, ψ)]dψ (33)

Finally, under this standard approach, surrogate data such as random permutations are used to assess the statistical significance of the observed MI. Random time shifts Δt are drawn from a uniform distribution whose interval depends on the problem dynamics and phase amplitude coupling is estimated using the shifted fast amplitudes A_{t-Δt}^{f} and the original slow phase ϕₜ^{s}. The MI is then calculated for this permuted time series, and the process is repeated to construct a null distribution for the MI. The original MI is deemed significant if it is bigger that 95% of the permuted values. Overall, this method requires that the underlying process remains stationary for sufficiently long so that the modulogram can be estimated reasonably well and so that enough comparable data segments can be permuted in order to assess significance.

Instead, we introduce a parametric representation of cross-frequency analysis that can quantify the cross-frequency coupling relationship with a smaller number of parameters. To do so, we consider a constrained linear regression problem of the form Aₜ^{f}=X(φ)β+ϵₜ which can ultimately be rewritten:

Aₜ^{f}=A₀[1+K^{mod} cos(ϕₜ^{s}−ϕ^{mod})]ϵₜ, ϵₜ˜ [symbol] (0, σ_{β}²) (34)

K^{mod} controls the strength of the modulation while ϕ^{mod} is the preferred phase around which the amplitude of the fast oscillation xₜ^{f} is maximal as shown in FIG. 12H-J. FIG. 12A shows raw EEG data. FIG. 12B shows a six second window of the EEG data. FIG. 12C shows a slow oscillation fit in the six second window. FIG. 12D shows a fast oscillation fit in the six second window. FIG. 12E shows an exemplary form of the state space model. FIG. 12F shows slow oscillation phase. FIG. 12G shows fast oscillation amplitude. FIG. 12H shows estimated K^{mod} and the distribution. FIG. 12I shows estimated ϕ^{mod} and the distribution. FIG. 12J shows regressed alpha wave amplitude. For example, if K^{mod}=1 and ϕ^{mod}=0, the fast oscillation is strongest at the peak of the slow oscillation. On the other hand, if ϕ^{mod}=π, the fast oscillation is strongest at the trough or nadir of the slow oscillation.

Finally, instead of relying on surrogate data to determine statistical significance, which decreases efficiency even further, our model formulation allows us to estimate the posterior distribution of the modulation parameters p (K^{mod}, ϕ^{mod}|{yₜ}ₜ) and to deduce the associated credible intervals (CI) as shown in FIG. 12F-J.

We refer to our approach as the state-space cross-frequency analysis (SSCFA) method. Because physiological systems are time varying, we apply it over multiple non-overlapping windows. In a variation of our method, we can also impose a temporal continuity constraint on the modulation parameters across windows using a second state-space model, yielding what we term the double state-space cross-frequency analysis estimate (dSSCFA).

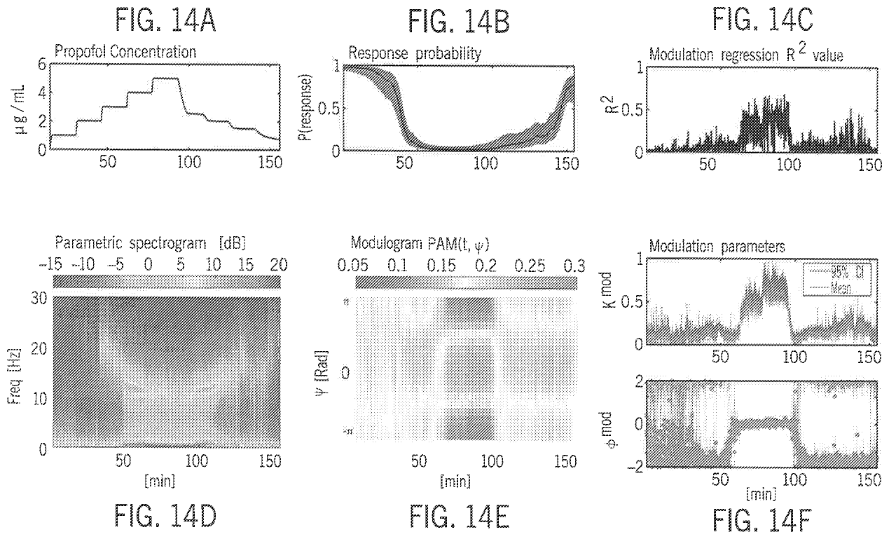

To demonstrate the performance of our methods we first analyzed EEG data from a human volunteer receiving propofol to induce sedation and unconsciousness as shown in FIG. 14. FIG. 14A shows propofol concentration supplied to the patient EEG data. FIG. 14B shows a response probability of the patient EEG data. FIG. 14C shows modulation regression values of the patient EEG data. FIG. 14D shows a parametric spectrum of the patient EEG data. FIG. 14E shows a modulogram PAM of the patient EEG data. FIG. 14F shows modulation parameters of the patient EEG data. As expected, as the concentration of propofol increases, the subject's probability of response to auditory stimuli decreases. The parametric power spectral density (see equation (59) changes during this time, developing beta (12.5-25 Hz) oscillations as the probability of response begins to decrease, followed by slow (0.1-1 Hz) and alpha (8-12 Hz) oscillations when the probability of response is zero as shown in FIG. 14D. For a window T, we estimate the modulation strength K^{mod} and phase ϕ_{T}^{mod} (and CI) with dSSCFA as shown in FIG. 14F and we gather those estimates in the Phase Amplitude Modulogam: PAM(T,) of FIG. 14E. For a given window T, PAM(T, .) is a probability density function (pdf) having support [−π, π]. It assesses how the amplitude of the fast oscillation is distributed with respect to the phase of the slow oscillation. When the probability of response is zero, we observe a strong “peakmax” (K^{mod}≠0.8, ϕ_{T}^{mod}≈0) pattern in which the fast oscillation amplitude is largest at the peaks of the slow oscillation. During the transitions to and from unresponsiveness, we observe a “troughmax” pattern of weaker strength (K^{mod}≠0.25, ϕ_{T}^{mod}=±π) n which the fast oscillation amplitude is largest at the troughs of the slow oscillation. Note that the coefficient of determination R² for the modulation relationship mirrors the coupling strength K^{mod} since Aₜ^{f} is better predicted by our model when the coupling is strong.

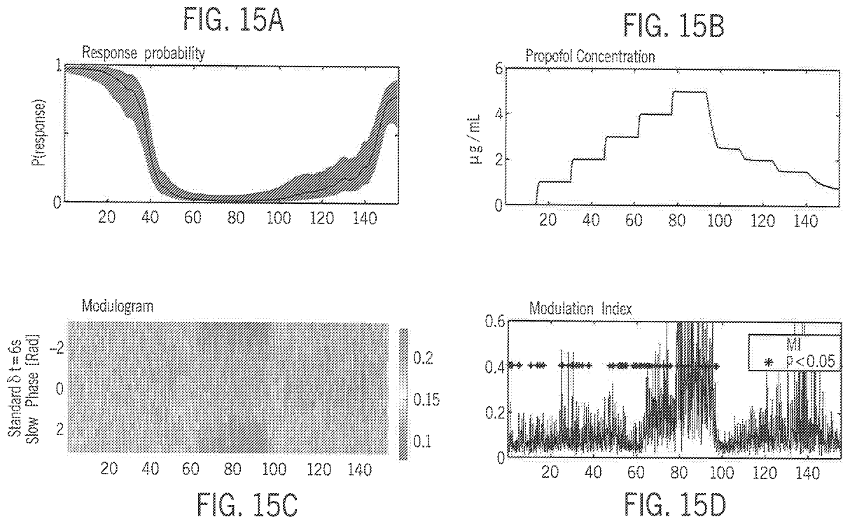

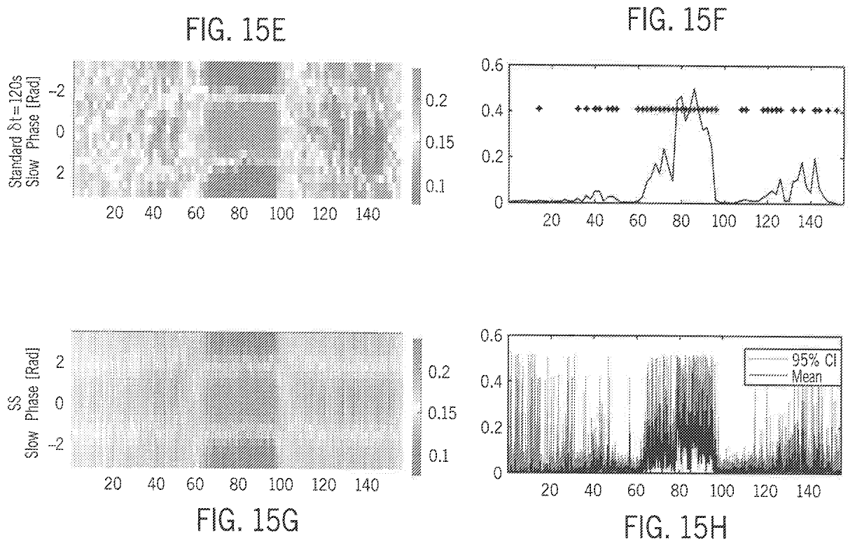

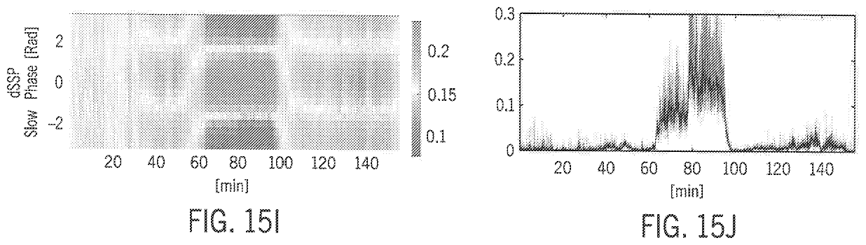

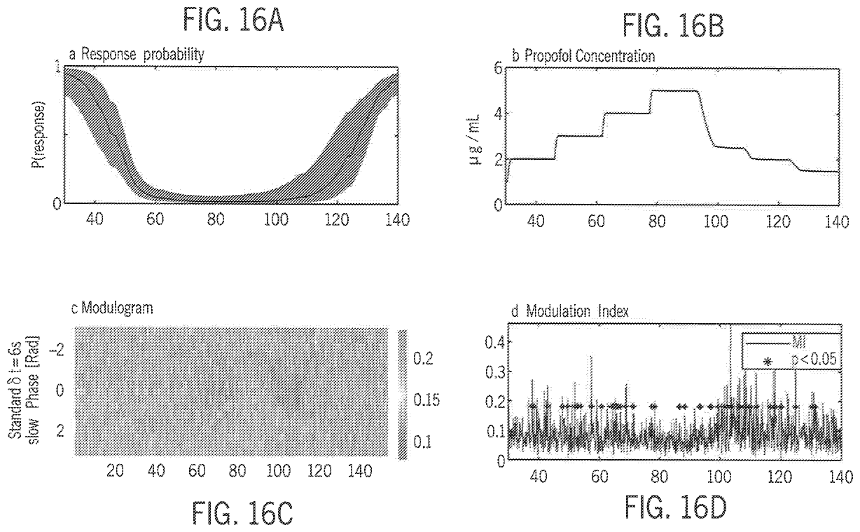

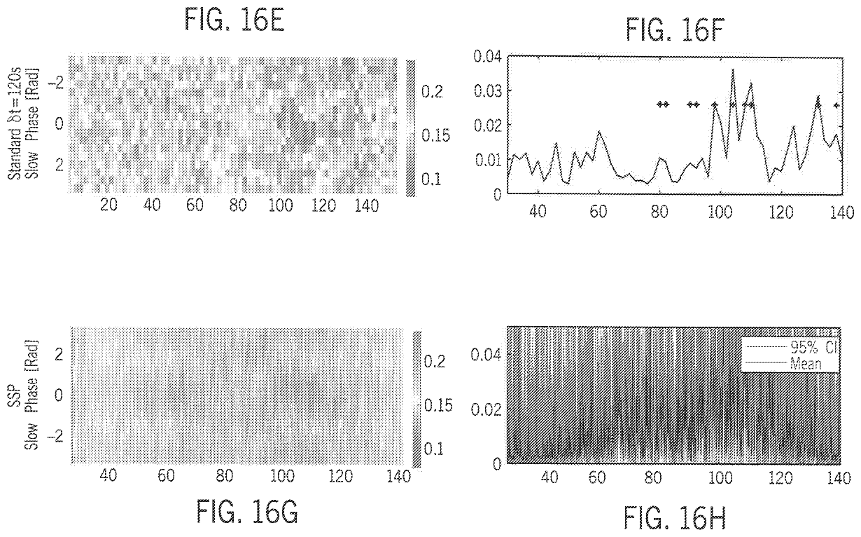

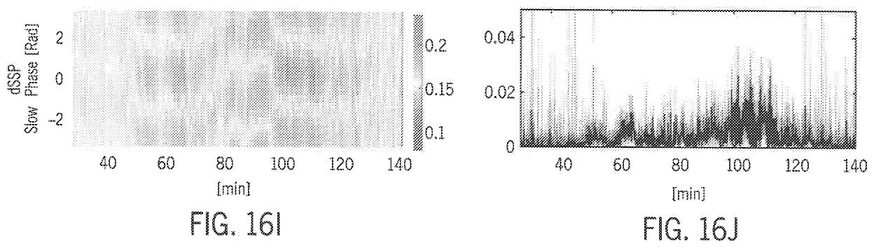

When averaged over long, continuous and stationary time windows, conventional methods provide good qualitative assessments of PAC. However, in many cases, analyses over shorter windows of time may be necessary if the experimental conditions or clinical situation changes rapidly. In previous work, PAC has been analyzed using conventional methods with relatively long δt=120 s windows, appropriate in this case because propofol was administered at fixed rates over ˜14 minute intervals. The increased statistical efficiency of the SSCFA and dSSCFA methods makes it possible to analyze much shorter time windows of δt=6 s, which we illustrate in two subjects, one with strong coupling (shown in FIG. 15) and another with weak coupling (shown in FIG. 16). FIG. 15A is a response probability plot. FIG. 15B is a propofol concentration plot. FIG. 15C is a modulogram for a standard approach with six second time windows. FIG. 15D is a modulation index plot associated with FIG. 15C. FIG. 15E is a modulogram for a standard approach with one hundred and twenty second time windows. FIG. 15F is a modulation index plot associated with FIG. 15E. FIG. 15G is a modulogram for the SSCFA approach. FIG. 15H is a modulation index plot associated with FIG. 15G. FIG. 15I is a modulogram for the dSSCFA approach. FIG. 15J is a modulation index plot associated with FIG. 15I. FIG. 16A is a response probability plot. FIG. 16B is a propofol concentration plot. FIG. 16C is a modulogram for a standard approach with six second time windows. FIG. 16D is a modulation index plot associated with FIG. 16C. FIG. 16E is a modulogram for a standard approach with one hundred and twenty second time windows. FIG. 16F is a modulation index plot associated with FIG. 16E. FIG. 16G is a modulogram for the SSCFA approach. FIG. 16H is a modulation index plot associated with FIG. 16G. FIG. 16I is a modulogram for the dSSCFA approach. FIG. 16J is a modulation index plot associated with FIG. 16I.

To do so, we compare SSCFA, dSSCFA and standard methods used with δt=120 s or δt=6 s based on the modulogram and on the Modulation Index (MI) estimates. The latter assesses the strength of the modulation by measuring, for any window T how different PAM(T, .) is from the uniform distribution. The Kullback-Leibler Divergence is typically used for this purpose. Thus, any random fluctuations in the estimated PAM will increase MI, introducing a bias. Our model parametrization is used to derive PAM, MI and associated CI but standard non-parametric analysis typically rely on binned histogram. As a results they estimate statistical significance by constructing surrogate datasets and reporting p-values.

Both subjects exhibit the typical phase-amplitude cross correlation profile previously described when they transition in and out of unconsciousness. Nevertheless, since SSCFA more efficiently estimates phase and amplitude and produces smooth PAM estimates even on short windows, MI estimates derived from SSCFA show less bias that the standard approach. For the same reasons, mod estimates show less variance than the standard approach. The dSSCFA algorithm provides a temporal continuity constraint on the PAM, making it possible to track time-varying changes in PAC while further reducing the variance of the PAM estimates. Finally, our parametric strategy provides posterior distributions for K^{mod}, ϕ^{mod} and MI, making it possible to estimate CI for each variable and it assesses significance without resorting to surrogate data methods.

To illustrate the performance of our approach in a different scenario representative of invasive recordings in animal models, we analyzed rat data during a learning task hypothesized to involve theta (6-10 Hz) and low gamma (25-60 Hz) oscillations. We applied dSSCFA on 2 second windows and confirmed that theta-gamma coupling in the CA3 region of the hippocam pus increases as the rat learned the discrimination task. In our analysis using dSSCFA, we did not pre-select the frequencies of interest, nor did we specify bandpass filtering cutoff frequencies. Rather, the EM algorithm was able to estimate the specific underlying oscillatory frequencies for phase and amplitude from the data, given an initial starting point in the theta and gamma ranges. Thus we illustrate that our method can be applied effectively to analyze LFP data, and that it can identify the underlying oscillatory structure without having to specify fixed frequencies or frequency ranges.

To test our algorithms in a more systematic way as a function of different modulation features and signal to noise levels, we analyzed multiple simulated data sets. By design, these simulated data were constructed using generative processes or models different than the state space oscillator model; i.e., the simulated data generating processes were outside the “model class” used in our methods. Here, we focus on a slow component and an alpha component to reproduce our main experimental data cases. In doing so, our intent is not to provide an exhaustive characterization of the precision and accuracy of our algorithm, since this would strongly depend on the signal to noise ratio, the signal shape, etc. Instead, we aim to illustrate how and why our algorithm outperforms standard analyses in the case of short and noisy time-varying data sets.

We first compare the resolution and robustness of dSSCFA with conventional techniques on broad-band signals with modulation parameters varying on multiple time scales. Results were reported for different generative parameters (See further below, Δfₛ^{gen}=Δf_{f}^{gen}, σₛ and σ_{f} and associated signal. Although robust when averaged on long windows with stationary coupling parameters, standard techniques become ineffective when the modulation parameters vary rapidly across windows. The modulation cannot be resolved when long windows are used. However if we reduce the window size to compensate, the variance of the estimates increases significantly. A trade-off has to be found empirically. On the other hand, we see that, applied on 6-second windows, (d)SSCFA can track the rapid changes in amplitude modulation even in the case of a low signal to noise ratio. The dSSCFA algorithm also provides estimates of the posterior distribution of the modulation parameters, making it straightforward to construct CI and perform statistical inference. By comparison, the surrogate data approach becomes infeasible as there are fewer and fewer data segments to shuffle.

In one study, a nonlinear PAC formulation was described as a driven autoregressive (DAR) process, where the modulated signal is a polynomial function of the slow oscillation. The latter, referred to as the driver, is filtered out from the observation around a preset frequency and used to estimate DAR coefficients. The signal parametric spectral density is subsequently derived as a function of the slow oscillation. The modulation is then represented in terms of the phase around which the fast oscillation power is preferentially distributed. A grid search is performed on the driver, yielding modulograms for each slow central frequency over a range of fast frequencies. The frequencies associated with the highest likelihood and/or strongest coupling relationship are then selected as the final coupling estimate.

This parametric representation improves efficiency, especially in the case of short signal windows, but because it relies on an initial filtering step, it also shares some of the limitations of conventional techniques. As we will see, spurious CFC can emerge from abruptly varying signals or nonlinearities. Additionally, this initial filtering step might contaminate PAC estimates from short data segments with wideband slow oscillations.

To compare our methods with standard techniques and the DAR method, we generated modulated signals (equation (88), λ=3, and ϕ^{mod}==π/3) using different frequencies of interest (fₛ and f_{f}) spectral widths (Δfₛ^{gen}) and Signal to Noise Ratios (SNR). Typical signal traces for those generating parameters can be generated using equation (87). We then compare how well these methods recover the slow oscillation and the fast oscillation, which or the modulation phase. Contrary to the other methods presented here, SSCFA does not compute the full comodulograms to select frequencies of interest but rather identifies them by fitting the state space oscillator model. Coupling is only estimated in a second step. Although we used tangible prior knowledge in previous sections to initialize the algorithm, we adapt an initialization procedure to provide a fair comparison. For each condition, we generated 400 six-second windows. When necessary, the driver was extracted using Δfₛ^{filt}=Δfₛ^{gen}.

We found that our algorithm better retrieves fast frequencies in each case especially when the slow oscillation is wider-band. It also outperforms the other methods when estimating modulation phase: our algorithm is stable in the case of broadband (Δfₛ^{gen}=3 Hz) or weak (σₛ,σₛ)=(0.7, 0.3)) slow oscillations and ϕ^{mod} is estimated accurately with very few outliers and a smaller standard deviation in virtually all cases considered.

Despite the central role that CFC likely plays in coordinating neural systems, standard methods of CFC analysis are subject to many caveats that are a source of ongoing concern. Spurious coupling can arise when the underlying signals have sharp transitions or nonlinearities. On the other hand, true underlying coupling can be missed if the frequency band for bandpass filtering is not selected properly. Here we illustrate how our SSCFA method is robust to all of these limitations. We also show how our method is able, counterintuitively, to extract nonlinear features of a signal using a linear model.

Oscillatory neural waveforms may have features such as abrupt changes or asymmetries that are not confined to narrow bands. In such cases, truncating their spectral content with standard bandpass filters can distort the shape of the signal and can introduce artefactual components that may be incorrectly interpreted as coupling.

The state space oscillator model provides an alternative to bandpass filtering that can accommodate non sinusoidal wave-shapes. In this section, we extend the model to explicitly represent the slow oscillatory signal's harmonics, thus allowing the model to better represent oscillations that have sharp transitions and those that may be generated by nonlinear systems. To do so, we optimize h oscillations with respect to the same fundamental frequency fₛ, which is further described below. We further combine this model with information criteria (Akaike Information Criteria-AIC- or Bayesian Information Criteria-BIC-) to determine (i) the number of slow harmonics h and (ii) the presence or the absence of a fast oscillation. We select the best model by minimizing ΔIC=IC−min(IC). We only report AIC based PAC estimation here although both AIC and BIC perform similarly. When multiple slow harmonics are favored, we use the phase of the fundamental oscillation to estimate PAC.

We first simulated a non symmetric abruptly varying signal using a Van der Pol oscillator (equation (89), ϵ=5, ω=5s⁻¹) to which we added observation noise (vₜ˜ [symbol] (0, R), \sqrt{R}=0.15). We then considered two scenarios, one with a modulated fast sinusoidal wave (FIG. 27A, Aₜ^{f}=A₀(1+cos ϕₜ^{s}), A₀=2\sqrt{R} and f_{f}=10 Hz), and one without (FIG. 27B). Because our model is able to fit the sharp transitions, both AIC and BIC identify the correct number of independent components. As a consequence, when no clear fast oscillation is detected, no PAC is calculated. On the other hand, when no fast oscillation is present, standard techniques extract a fast component stemming from the abruptly changing slow oscillation, leading to the detection of spurious coupling.

Nonlinear inputs arising from signal transduction harmonics are a similar hurdle in CFC analysis. If we consider a slow oscillation xₜ^{s}=cos(ωₛt) non-linearly transduced as yₜ=g(xₜ^{sl ), we can write a second order approximation:}

yₜ≈xₜ^{s}+a(xₜ^{s})²=cos(ωₛt)+a[1+cos(2ωₛt)]/2 (35)

If

$$\frac{{w}_{s}}{2 π} = 1 Hz ,$$

bandpass filtering yₜ around 0.9-3.1 Hz to extract an oscillation peaking at f_{f}=2 Hz would yield:

x_{f}^{f}=(1+a cos(ωₛt))cos(ωₛt) (36)

In such a case, standard CFC analysis infers significant coupling while oscillation decomposition correctly identifies a harmonic decomposition without CFC.

We observed that the (extended) state-space oscillator is better suited to model physiological signals than narrow band components. In addition, the model selection paradigm combined with prior knowledge of the signal content (e.g., propofol anesthesia slow-alpha or rodent hippocampal theta-gamma oscillations) allows us to study cross-frequency analysis in a more principled way.

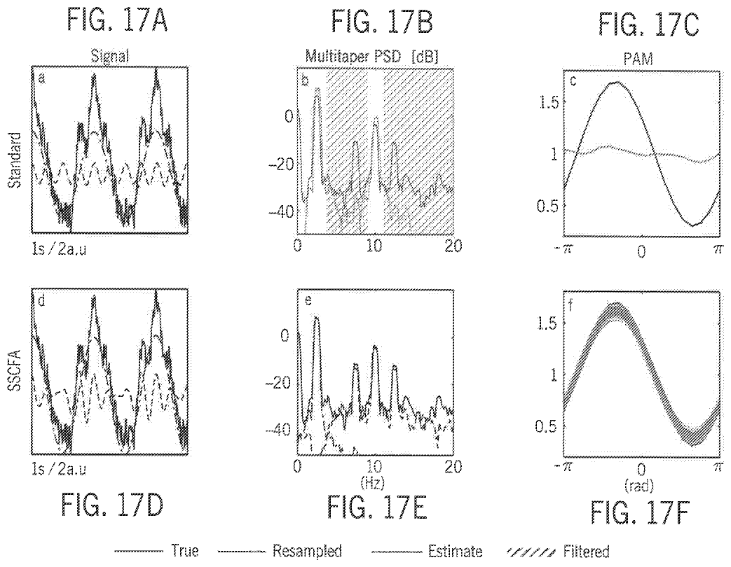

If bandpass filters with an excessively narrow bandwidth are applied to a modulated signal, the modulation structure can be obliterated. Our SSCFA algorithm uses a state-space oscillator decomposition which does not explicitly model the structural relationship giving rise to the modulation side lobes. During testing, we found that the modulation is successfully extracted, as observed in fitted time series and in the spectra. FIG. 17A shows decomposition of components of oscillatory components using a standard method, while FIG. 17D shows decomposition of components of oscillatory components using the SSCFA method for a given signal. FIG. 17B shows power spectral density determined using the standard method, while FIG. 17E shows power spectral density determined using the SSCFA method. FIG. 17C shows a phase-amplitude modulation estimate by a standard method while FIG. 17F shows a phase-amplitude modulation estimate by the SSCFA method for the given EEG signal. The oscillation decomposition method used by the SSFCA method is the same as the oscillation decomposition method used by the dSSFCA method. The model is able to achieve this by making the frequency response of the fast component wide enough to encompass the side lobes. The algorithm does this by inflating the noises covariances R and σ_{f}² and σ_{f}² and deflating a_{f}. It might be possible to use a higher order model like an ARMA(4,2) (which would represent the product of 2 oscillations and which poles are in aₛa_{f}e^{±i(w}^{_{f}}^{±w}^{ₛ}⁾, or to directly model coupling through a nonlinear observation. However, in both cases, we found that such models were difficult to fit to the data, and quickly became underconstrained when applied to noisy, non-stationary, non-sinusoidal physiological signals. Instead, we found that our simpler model was able to pull out the modulated high-frequency component robustly.

Although the EM algorithm ensures convergence, the log likelihood which is to be maximized is not always concave. A signal composed of d oscillations can be initialized with the parameters of the best autoregressive (AR) process of order p∈[|d,2d|]. Nevertheless, because of the electrophysiological signal's aperiodic component, such procedure might bias the initialization. Indeed, the aperiodic components are usually described by a 1/f^{x} power-law function, which might be regressed by the AR process. In such cases, the initialization could fail to account for an actual underlying oscillation.

To help mitigate this potential problem, we adapt Halleret al.'s algorithm to the state space oscillation framework. Our initialization algorithm aims to disentangle the oscillatory components from the aperiodic one before fitting the resulting spectra with the parametric power spectral density (PSD) of the oscillation (equation (ZZHH)), which is described further below.

The PSD for the observed data signal yₜ can be estimated using the multitaper method. The frequency resolution r_{f} is then set, typically to 1 Hz, which yields the time bandwidth product

$$TW = \frac{{r}_{f}}{2} \frac{N}{{F}_{s}} .$$

The number of taper K is then chosen such that K«└2TW┘−1.

The observation noise R₀ (used to initialized R) can be estimated using:

$$10 {log}_{10} \frac{{R}_{0}}{Fs} = {lim}_{f \to ∞} PSD ( f ) \qquad ( 37 )$$

The observation noise R₀ can then be removed from the PSD.

The non-oscillatory component can be regressed out from the PSD. The aperiodic signal PSD in dB, at frequencies f is then modeled by:

g(f)=g₀−log(1+(f/f₀)^{x}) (38)

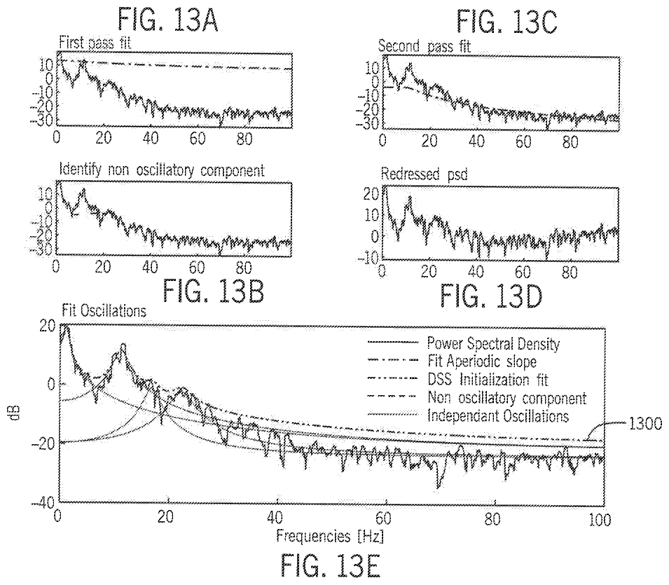

X controls the slope of the aperiodic signal, g₀ the offset and f₀ the “knee” frequency. A first pass fit is applied to identify the frequencies corresponding to non oscillatory components: only f₀ is fitted while X and g₀ are respectively set to X=2 and g₀=PSD(f=0), as shown in FIG. 13A. A threshold is fixed (typically 0.8 quantile of the residual) to identify frequencies associated to the aperiodic signals, as shown in FIG. 13B. A second pass fit is then applied only on those frequencies from which we deduce f₀, x, and g₀ as shown in FIG. 13C. We remove g(f) from the raw PSD in dB to generate a redressed PSD for the second step of the algorithm as shown in FIG. 13D. The fitted parameters can then be used to initialize the EM algorithm.

From the redressed PSD, we fit a given number d₀ (e.g d₀=4) of independent oscillations using the theoretical PSD given in equation (ZZHH). To do so, we identify PSD peaks of sufficient width (wider than r_{f}/2) before fitting an oscillation theoretical spectra in a neighborhood of width 2r_{f} around this peak. For oscillation j, we deduce (fⱼ)₀, (aⱼ)₀ and (\tilde{σ}ⱼ²)₀. Since (\tilde{σ}ⱼ²)₀ represents the offset of a given oscillation after removing the aperiodic component, we adjust it to estimate σⱼ²:

$$10 {log}_{10 } ( \frac{{( {\tilde{σ}}_{j}^{2} )}_{0}}{{F}_{s}} ) \approx 10 {log}_{10 } ( \frac{{( {σ}_{j}^{2} )}_{0}}{{F}_{s}} ) + g ( {( {f}_{j} )}_{0} ) \qquad ( 39 )$$

The resulting spectra PSDⱼ are then subtracted and the process is repeated until all oscillations are estimated as shown by DSS initialization fit line 1300 in FIG. 13E. We finally estimate the power Pⱼ of an oscillation j in the neighborhood of (fⱼ)₀ and estimate its contribution to the total power P₀ by:

$$- 10 {log}_{10 } ( 1 - \frac{{P}_{j}}{{P}_{0} + 2 {r}_{f} {R}_{0} \text{/} {F}_{s}} ) . \qquad ( 40 )$$

Oscillations are sorted and the resulting parameters are used to initialize the EM algorithm with the d∈[|1, d₀|] first oscillations.

To improve statistical efficiency, we introduce a parametric representation of PAC. For a given window, we consider the following (constrained) linear regression problem:

$$\begin{matrix} \{ \begin{matrix}{A}_{t}^{f} & = X ( {ϕ}_{t}^{s} ) β + {∈}_{t} , {∈}_{t} ∼ ( 0 , {σ}_{β}^{2} ) \\ β & ∈ W ( \overline{K} )\end{matrix} \\ where β = {[ {β}_{0} {β}_{1} {β}_{2} ]}^{T} , X ( {ϕ}_{t}^{s} ) = [ 1 cos ( {ϕ}_{t}^{s} ) sin ( {ϕ}_{t}^{s} ) and W ( \overline{K} ) = [ β ∈ {ℝ}^{3} \vert \sqrt{{β}_{1}^{2} + {β}_{2}^{2}} < {β}_{0} \overline{K} ] . If we define \text{:} & ( 41 ) \\ {K}^{mod} = \sqrt{{β}_{1}^{2} + {β}_{2}^{2}} \text{/} {β}_{0} , {ϕ}^{mod} = {tan}^{- 1} ( {β}_{2} \text{/} {β}_{1} ) , and {A}_{0} = {β}_{0} & ( 42 )\end{matrix}$$

we see that equation (41) is equivalent to:

$$\{ \begin{matrix}{A}_{t}^{f} = {A}_{0} [ 1 + {K}^{mod} cos ( {ϕ}_{t}^{s} - {ϕ}^{mod} ) \vert + {∈}_{t} , {∈}_{t} ∼ ( 0 , {σ}_{β}^{2} ) \\ {K}^{mod} ∈ [ 0 , \vec{K} )\end{matrix} \qquad ( 43 )$$

Setting K̅=1 ensures that the model is consistent, i.e., that the modulation envelope cannot exceed the amplitude of the carrier signal. But this can be a computationally expensive constraint to impose. If the data have a high signal to noise ratio so that K^{mod} is unlikely to be greater than 1 by chance, we could also choose to solve the unconstrained problem (K̅=±∞). Under the constrained (44) the posterior distribution for β is a truncated multivariate t-distribution:

$$p ( β \vert \{ {A}_{t}^{f} , {ϕ}_{t}^{s} \} t ) = \frac{1}{Z} {( 1 + {{v}^{- 1} ( β - \overline{β} )}^{T} ( V \text{/} b ) ( β - \overline{β} ) )}^{- \frac{v + 3}{2}} {\{ β ∈ W ( \overline{K} ) \}}_{}$$

The likelihood, conjugate prior, posterior parameters β, V, b, v, and the normalizing constant Z are justified and derived further below. We refer to this estimate as state space cross frequency analysis (SSCFA) and we note:

β^{SAP}=argmax p(β|{Aₜ^{f}, ϕₜ^{N}}ₜ). (45)

The standard approach relies on surrogate data to determine statistical significance, which decreases its efficiency even further. Instead, we estimate the posterior distribution p(β|{yₜ}ₜ) from which we obtain the credible intervals (CI) of the modulation parameters K^{mod} and ϕ^{mod}. To estimate the posterior distribution, we sample from the posterior distributions given by (i) the state space oscillator model and (ii) the parametric PAC model described above.

(i) The Kalman Filter used in the r^{th} E-Step of the EM algorithm described above provides the following moments, for t, t′=1 . . . N:

xₜ^{N}= [symbol] ᵣ(xₜ|{yₖ}ₖ₌₁^{N}{ₖ₌₁^{N}), P_{t,t′}^{N}=covᵣ(xₜ, x_{t′}|{yₖ}ₖ₌₁^{N}) (46)

Therefore, we can sample l₁ times series: X={Xₜ}ₜ₌₁^{N} using:

X|{yₜ}ₜ₌₁^{N}˜ [symbol] ({xₜ^{N}}ₜ₌₁^{N}, P) (47)

where P is a 4N×4N matrix whose block erntries are given by (P)_{u′}P_{t,t′}^{N},

(ii) For each X, we use equation (8) to compute the resampled slow oscillation's phase ϕ and fast oscillation's amplitude [symbol] . We then use equation (44) to draw l₂ samples from p (β|, [symbol] ϕ). As a result, we produce l₁×l₂ samples to estimate:

$$\begin{matrix}p ( β \vert {\{ {y}_{t} \}}_{t} ) = {\int }_{𝒳} p ( β \vert 𝒳 ) p ( 𝒳 \vert {\{ {y}_{t} \}}_{t} ) \\ = p ( β \vert , φ ) p ( , φ \vert {\{ {y}_{t} \}}_{t} )\end{matrix} \qquad ( 48 )$$

We finally construct CI around β^{SSCFA} using an L₂ norm and in turn derive the CI of K^{mod} and ϕ^{mod}

We segment the time series into multiple non-overlapping windows of length N to which we apply the previously described analysis. We hence produce {β^{SSCFA}}_{T}, a set of vectors in [symbol] ³ accounting for the modulation where T denotes a time window of length N. or the modulation where T denotes a time window of length N.

A second state-space model can be used to represent the modulation dynamics. Here we fit an autoregressive (AR) model of order p with observation noise to the modulation vectors β^{SSCFA} across time windows. It yields the double state space cross frequency analysis estimate (dSSCFA):

β_{T}^{SSP}=β_{T}^{SSP}+γ_{T}, γ_{T}˜ [symbol] (0, R_{β})

β_{T}^{dSSP}=Σₖ₌₁^{p}hₖβ_{T-1}^{dSSP}+η_{T}, η_{T}˜ [symbol] (0, Q_{β}) (49)

We proceed by solving and optimizing Yule-Walker type equations numerically, which will be described below, and we select the order p with Bayesian information criterion. Finally, we can use the fitted parameters to filter the l₁×l₂resampled parameters to construct a CI for {β^{SSCFA}}_{T} when necessary.

To better compare standard techniques with the SSCFA, we derive an approximate expression for the PAM under our parameteric model for a window T:

$$P A M ( T , ψ ) = \frac{1}{2 π} ( 1 + \frac{sin ( δψ \text{/} 2 )}{δψ \text{/} 2} {K}_{T}^{mod} cos ( ψ - {ϕ}_{T}^{mod} ) ) {\to }_{δψ \to 0} \frac{1}{2 π} ( 1 + {K}_{T}^{mod} cos ( ψ - {ϕ}_{T}^{mod} ) ) \qquad ( 50 )$$

We analyzed human EEG data during loss and recovery of consciousness during administration of the anesthetic drug propofol. Briefly, 10 healthy volunteers (18-36 years old) were infused increasing amounts of propofol spanning 6 target effect site concentrations (0, 1, 2, 3, 4, and 5 μg.L⁻¹). Infusion was computer controlled and each concentration was maintained for 14 minutes. To monitor loss and recovery of consciousness behaviorally, subject were presented with an audio stimulus (a click or a verbal command) every 4 seconds and had to respond by pressing a button. The probability of response and associated 95% credible intervals were estimated using Monte-Carlo methods to fit a state space model to these data. Finally, EEG data were pre-processed using an anti-aliasing filter and downsampled to 250 Hz.

We computed spectrograms of the EEG using the parametric expression associated with oscillation decomposition. Standard techniques for PAC analysis were applied on 6 and 120 second windows for which alpha and slow component were assumed to be known and extracted using bandpass filters around 0.1-1 Hz and 9-12 Hz. Significance for the standard PAC method was assessed using 200 random permutations.

Below, we derive the parametric expression for the power spectral density (PSD) of an oscillation x_{1,t}^{j}, by building an autoregressive moving average process (ARMA) with the same spectral content. For convenience, we will drop the index j in what follows. First, we note that an oscillation is asymptotically second order stationary. Let us compute its autocovariance sequence. Since [symbol] is a rotation matrix [symbol] (ω)^{k}= [symbol] (kω) and [symbol] [symbol] ^{T}=I. Therefore, from equation (2), it comes:

$$\begin{matrix}\begin{matrix}𝔼 ( {x}_{t} {{x}_{t}}^{T} ) = a ( ω ) 𝔼 ( {x}_{t - 1} {{x}_{t - 1}}^{T} ) {( a ( ω ) )}^{T} + Q \\ = Q {\sum }_{t = 0}^{N} {a}^{2 t} \\ = Q \text{/} ( 1 - {a}^{2} ) + 𝒪 ( {a}^{2 N} )\end{matrix} \\ and \text{:} & ( 51 ) \\ \begin{matrix}𝔼 ( {x}_{t + k} {{x}_{t}}^{T} ) = a ( ω ) 𝔼 ( {x}_{t + h - 1} {x}_{t} ) \\ = {a}^{k} ( k ω ) Q \text{/} ( 1 - {a}^{2} ) + 𝒪 ( {a}^{2 N + k} )\end{matrix} & ( 52 )\end{matrix}$$

We can hence write, for t=1 . . . N, k=0 . . . N−t,s_{t,t+k},= [symbol] (x_{1,t+k}x₁,t)=sₖ. As a consequence, an oscillation can be approximated by a second order stationary process, and in virtue of the Wiener-Khinchin theorem, its theoretical power spectral density is:

$$S ( f ) = {lim}_{N \to ∞} \frac{1}{{F}_{s}} ( {s}_{0} + 2 {\sum }_{t = 1}^{N} {s}_{t} {e}^{- 2 i π tf \text{/} {F}_{s}} ) \qquad ( 53 )$$

We now consider the ARMA(2,1):

\tilde{x}ₜ=ϕ₁\tilde{x}ₜ₋₁+ϕ₂\tilde{x}ₜ₋₂+ũ+ψ₁ũₜ₋₁, ũₜ˜ [symbol] (0, \tilde{σ}²) (54)

to which we impose, for t=1 . . . N, k=0 . . . N−t: [symbol] (\tilde{x}ₜ\tilde{x}ₜ₊ₕ¹)=sₖ. It follows that:

$${s}_{k} = {ϕ}_{1} {s}_{k - 1} + {ψ}_{2} {s}_{k - 2} , k > 2 \\ {s}_{1} = \frac{{ϕ}_{1} {s}_{0} + {ψ}_{1} {\tilde{σ}}^{2}}{1 - {ϕ}_{2}} \\ {s}_{0} = {ϕ}_{1} {s}_{1} + {ϕ}_{2} {s}_{2} + {\tilde{σ}}^{2} ( 1 + {ϕ}_{1} {ψ}_{1} + {ψ}_{1}^{2} ) \qquad ( 55 )$$

### Taking

ϕ₁=2a cos(ω)

ϕ₂=−a² (56)

satisfies the first equality of equation (55). The remaining conditions can then be rewritten:

$${1}^{{\tilde{σ}}^{2}} = - a {σ}^{2} cos ( ω ) \\ 0 = {ψ}_{1}^{2} + \frac{1 + {a}^{2}}{acos ( ω )} {ψ}_{1} + 1 \qquad ( 57 )$$

from which we deduce 2 two negative roots ψ₁^{±} ultimately leading to the same autocovariance series. Overall, we choose:

$${ψ}_{1} = - \frac{1}{2} ( \frac{1 + {a}^{2}}{a cos ( ω )} + \sqrt{{( \frac{1 + {a}^{2}}{a cos ( ω )} )}^{2} - 4} ) \\ {\tilde{σ}}^{2} = - \frac{a {σ}^{2} cos ( ω )}{1} \qquad ( 58 )$$

Applying the discrete Fourier transform to equation (54) yields:

[symbol]

[\tilde{x}](f)(1−ϕ₁e^{-2iπf/Fs}−ϕ₂e^{-4iπf/Fs})= [symbol] [ũ](f)(1+ψ₁e^{-2iπf/Fs}) (54B)

since ũₜ is Gaussian noise, [symbol] (ũₜuₜ₊ₕ^{T})=δₖδ². Therefore, our ARMA (2,1) PSD is:

$$S [ \tilde{x} ] ( f ) = \frac{{\tilde{σ}}^{2}}{{F}_{s}} \frac{{\vert 1 + {ψ}_{1} {e}^{- 2 i π f \text{/} {F}_{s}} \vert}^{2}}{{\vert 1 - {ϕ}_{1} {e}^{- 2 i π f \text{/} {F}_{s}} - {ϕ}_{2} {e}^{- 4 i π f \text{/} {F}_{s}} \vert}^{2}}$$

### (54C)

Finally, the PSD an oscillation centered in f₀ is:

$$S [ {x}_{1 , t} ] ( f ) = \frac{{\tilde{σ}}^{2}}{{F}_{s}} \frac{{\vert 1 + {ψ}_{1} {e}^{- 2 i π f \text{/} {F}_{s}} \vert}^{2}}{{F}_{s} {\vert ( {ae}^{- 2 i π f \text{/} {F}_{s}} - {e}^{- 2 i π {f}_{0} \text{/} {F}_{s}} ) ( {ae}^{- 2 i π f \text{/} {F}_{s}} - {e}^{+ 2 i π {f}_{0} \text{/} {F}_{s}} ) \vert}^{2}} \qquad ( 59 )$$

We use τ_{β}=σ_{β}⁻² and we assume that the likelihood of the model defined in equation (41) is:

$$p ( {A}^{f} , {ϕ}^{s} \vert β , {τ}_{β} ) ∝ {τ}_{β}^{- N \text{/} 2} exp ( - \frac{{τ}_{β}}{2} {( {A}^{f} - X ( {ϕ}^{s} ) β )}^{τ} ( {A}^{f} - X ( {ϕ}_{t}^{s} ) β ) ) {\{ β ∈ W ( \overline{K} ) \}}_{} \qquad ( 60 )$$

Where {Aₜ^{f}}ₜ=A^{f} and {ϕₜ^{s}}ₜ=ϕ^{s}. The conjugate prior is:

$$p ( β , {τ}_{β} ) ∝ {τ}_{β}^{\frac{b + 3}{2} - 1} exp ( - \frac{{τ}_{β}}{2} [ \tilde{b} \tilde{v} + {( β - \tilde{β} )}^{τ} \tilde{V} ( β - \tilde{β} ) ] ) {\{ β ∈ W ( \overline{K} ) \}}_{} \qquad ( 61 )$$

We choose prior hyperparameters \tilde{V}, \tilde{b}, \tilde{v} and \tilde{β}=[\tilde{β}₀\tilde{β}₁\tilde{β}₂]^{t} to convey as little information as possible on the phase and the strength of the modulation. Marginalizing equation (61) over τ_{β}, yields a truncated multivariate t-distribution:

$$p ( β ) ∝ {( 1 + \frac{1}{\tilde{v}} {( β - \tilde{β} )}^{τ} ( \tilde{V} \text{/} \tilde{b} ) ( β - \tilde{β} ) )}^{- \frac{b + s}{2}} {\{ β ∈ W ( \overline{K} ) \}}_{} \qquad ( 62 )$$

\tilde{v}≥3 insures that the multivariate-t variance is defined. It is \tilde{v}(\tilde{v}−2)⁻¹\tilde{b}\tilde{V}⁻¹. Then, we consider the independent random variables Ã, \tilde{K} and \tilde{ϕ} such that:

Ã˜Γ(A₀/c,c)

\tilde{K}˜Uniform(0,1)

\tilde{ϕ}˜Uniform(−π,π) (63)

We note γ=[Ã Ã\tilde{K} cos(ϕ̅) Ã\tilde{K} sin(\tilde{ϕ})]^{T}, use \tilde{b}=1, \tilde{v}=3 and we define \tilde{β}, and \tilde{V} such that:

\tilde{β}= [symbol] (γ) and \tilde{V}⁻¹=Cov(γ)(\tilde{v}−2)/\tilde{v} (64)

Additionally, we notice that if Aₜ^{f}≈A_{B[1+K}^{mod} cos(ϕₜ^{s}+ϕ^{mod})] and since [symbol] _{b} ( [symbol] cos(ϕₜ^{s}+ϕ^{mod)} [symbol] ₁)=0 (where [symbol] , represents an average over trials or windows and (.), is a temporal average across a given window), Eₛ ( [symbol] Aₜ^{t} [symbol] ₜ)=A₀ and [symbol] A[ [symbol] , ∈[0, 2A₀]. Therefore, it is reasonable to use A₀= [symbol] Aₜ^{f} [symbol] , and σ=1. Overall:

$$\tilde{v} = 3 , \tilde{V} = {〈 {A}_{t}^{f} 〉}_{t}^{- 1} ( \begin{matrix}3 & 0 & 0 \\ 0 & 12 & 0 \\ 0 & 0 & 12\end{matrix} ) \\ \tilde{β} = [ \begin{matrix}{〈 {A}_{t}^{f} 〉}_{t} \\ 0 \\ 0\end{matrix} ] and \tilde{b} = 1 \qquad ( 65 )$$

Posterior parameters are then given by:

v=\tilde{v}+N, V=\tilde{V}+X(ϕ^{s})^{T}X(ϕ^{s})

β̅V⁻¹(\tilde{V}\tilde{β}X(ϕ^{s})A^{f}), b=(\tilde{v}\tilde{b}+H)/v (66)

Where:

\hat{β}_{OLS}=(X(ϕ^{s})^{T}X(ϕ^{s}))⁻¹X(ϕ^{s})^{T}A^{t} and;

H=(A^{f}−X(ϕ^{s})\tilde{β}_{OLS})^{T}(A^{f}−X(ϕ^{s}\tilde{β}_{OLS})+(\hat{β}_{OLS}=β̅)^{T}X(ϕ^{s})(\hat{β}_{OLS}−β̅)+(\tilde{β}−β̅)^{T}\tilde{V}(\tilde{β}−β̅) (67)

We deduce the posterior distribution:

$$p ( β , {τ}_{β} \vert ( {A}_{t}^{f} , {ϕ}_{t}^{s} ) t ) = \frac{1}{Z} {τ}_{β}^{- \frac{\text{?}}{2} - 1} exp ( - \frac{{τ}_{β}}{2} [ bv + {( β - \overline{β} )}^{τ} V ( β - \overline{β} ) ] ) {( W β > 0 )}_{} \\ \text{?} \text{indicates text missing or illegible when filed} \qquad ( 68 )$$

The normalizing constant Z̅ is obtained by integration and is:

$$\overline{Z} = \frac{Γ ( v \text{/} 2 ) {( 2 π )}^{3 \text{/} 2}}{{\vert V \vert}^{1 \text{/} 2} {( vb \text{/} 2 )}^{v \text{/} 2}} P ( β ∈ W ( \overline{K} ) ) \qquad ( 69 )$$

where P(β∈W(K̅)) is computed using the multivariate t-distribution of parameters v, β̅ and b⁻¹V. Finally, we deduce equation (44) by marginilizing equation (68) over τ_{β} and have:

$$\overline{Z} = \frac{Γ ( v \text{/} 2 )}{Γ ( ( v + 3 ) \text{/} 2 )} \frac{{( v π )}^{3 \text{/} 2}}{{\vert V \text{/} b \vert}^{1 \text{/} 2}} \times P ( β ∈ W ( \overline{K} ) ) \qquad ( 70 )$$

Note that for large samples it might be useful to use:

$$\frac{Γ ( ( V + 3 ) \text{/} 2 )}{Γ ( v \text{/} 2 )} = ( \frac{v + 1}{2} ) {( \frac{v}{2} )}^{1 \text{/} 2} ( 1 - \frac{1}{4 v} + \frac{1}{32 {v}^{2}} + \frac{1}{128 {v}^{3}} + ( \frac{1}{{v}^{4}} ) ) \qquad ( 71 )$$

We now estimate parameters R_{β}, Q_{β}, and {hₖ}ₖ₌₁^{p} defined in equation (49) for a given AR order p. Let Cₘ= [symbol] (β^{SSCFA}, β_{T-m}^{SSCFA}^{^{T}}) be the autocovariance sequence of the modulation vectors estimated with equation (45). We have:

$${C}_{m} = {\sum }_{k = 1}^{v} {h}_{k} {C}_{m - k} + {R}_{β} ( {δ}_{0 , m} - {h}_{m} ) + Q {δ}_{0 , m} \qquad ( 72 )$$

where δ_{i,j} is the Kronecker delta. Equation (72) can be rewritten:

$$\{ \begin{matrix}{C}_{0} & = & {\sum }_{k = 1}^{p} {h}_{k} {C}_{k} + Q \\ [ \begin{matrix}{C}_{1} \\ {C}_{2} \\ ⋮ \\ {C}_{p}\end{matrix} ] & = & [ \begin{matrix}{C}_{0} - {R}_{β} & {C}_{1} & … & {C}_{p - 1} \\ {C}_{1} & {C}_{0} - {R}_{β} & … & {C}_{p - 2} \\ ⋮ & ⋮ & ⋱ & ⋮ \\ {C}_{p - 1} & {C}_{1} & … & {C}_{0} - {R}_{β}\end{matrix} ] [ \begin{matrix}{h}_{1} \\ {h}_{2} \\ ⋮ \\ {h}_{v}\end{matrix} ]\end{matrix} \qquad ( 73 )$$

For an observation noise candidate R*_{β}, if we can invert equation (73), we immediately access Q*_{β} and {h*ₖ}ₖ₌₁^{p}. Using the Kalman Filter, we hence deduce the likelihood of the candidate model.

Therefore, we note R_{β}^{m} the smallest eigenvalue of the Toeplitz matrix C=(C_{|i-j|})_{i,j=0 . . . p} and, numerically optimize the model likelihood with respect to R*_{β} in (0, R_{β}^{m}) where we know that (C−IR*_{β}) is full rank.

From R_{β} we get Q_{β} and {hₖ}ₖ₌₁^{p} then, once again, we use the Kalman Filter to estimate β_{T}^{dSSCFA}.

We now derive an approximated parametric modulogram for a window of length δt=N/F_{S} centered in τ. We will use:

$$l = {f}_{s} δ t \\ t = k \text{/} {F}_{s} \\ {Ω}_{τ} = \{ t , t ∈ [ τ - δ t \text{/} 2 , τ + δ t \text{/} 2 ] \} \\ {Ω}_{τ , ψ} = \{ t ∈ {Ω}_{τ} , {ϕ}_{t}^{s} ∈ [ ψ - \frac{δψ}{2} , ψ + \frac{δψ}{2} ] \} \\ {\tilde{Ω}}_{τ , ψ} \{ t ∈ {Ω}_{τ} , t {ω}_{s} ∈ [ ψ - \frac{δψ}{2} , ψ + \frac{δψ}{2} ] \} \qquad ( 74 )$$

For clarity we will use t or k without distinction and we remind that:

$$\begin{matrix} {A}_{k}^{f} = {A}_{0} [ 1 + {K}_{τ}^{mod} cos ( {ϕ}_{k}^{s} - {ϕ}_{τ}^{mod} ) ] + {∈}_{k} , {∈}_{k} ∼ ( 0 , {σ}_{β}^{2} ) & ( 75 ) \\ P A M ( τ , ψ ) = \frac{{\int }_{τ - δ t \text{/} 2}^{τ + δ t \text{/} 2} {\int }_{ψ - ψδ \text{/} 2}^{ψ + δψ \text{/} 2} {A}_{t}^{\text{?}} δ ( {ϕ}_{t}^{\text{?}} - {ψ}^{′} ) dtd {ψ}^{′}}{2 π {\int }_{τ - δ t \text{/} 2}^{τ + δ t \text{/} 2} {A}_{t}^{f} dt} = \frac{{P}_{2}}{{P}_{1}} \\ \text{?} \text{indicates text missing or illegible when filed} & ( 76 )\end{matrix}$$

Additionally, we assume that:
for

$$k ∈ {Ω}_{τ} , ϕ \text{?} = \frac{{ω}_{s}}{{F}_{s}} k + {η}_{k} , \\ \text{?} \text{indicates text missing or illegible when filed}$$

where

$$𝔼 ( {η}_{k} ) = 0 , Var ( {η}_{k} ) = {σ}_{ϕ}^{2} and {σ}_{ϕ}^{2} << \frac{{ω}_{s}}{{F}_{s}} < 1$$

and, for simplicity, for ψ∈[−π, π], for all h: [symbol] ⁺→ [symbol] smooth, Σ_{k∈}h(k)≈Σ_{k∈h(k) From the central limit theorem, Σ}_{kϵ}ₖ= [symbol] ₚ(\sqrt{N}) and Σ_{k∈η}ₖ= [symbol] ₚ(\sqrt{N}). We hence have:

$${P}_{1} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} + {η}_{k} ) ) + \frac{1}{{F}_{s}} {\sum }_{k ∈ \text{?}}^{ } {∈}_{k} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} ) ) + \frac{1}{{F}_{s}} {\sum }_{k ∈ Ω \text{?}}^{ } ( {∈}_{k} - {A}_{0} {K}_{τ}^{mod} sin ( \frac{{ω}_{s} k}{{F}_{s}} - {ϕ}_{τ}^{mod} ) {η}_{k} + \text{?} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} ) ) + ( \sqrt{N} ) \\ \text{?} \text{indicates text missing or illegible when filed} \qquad ( 77 )$$

### But

$${\sum }_{k ∈ Ω \text{?}}^{ } cos ( \frac{{ω}_{s} k}{{F}_{o}} - {ϕ}_{τ}^{mod} ) = cos ( \frac{\text{?}}{2 {F}_{s}} ( N - 1 ) - {ϕ}_{τ}^{mod} ) \frac{sin ( N \text{?} \text{/} ( 2 {F}_{0} ) )}{sin ( \text{?} \text{/} ( 2 {F}_{s} ) )} \leq \frac{2 {F}_{s}}{\text{?}} . \\ \text{?} \text{indicates text missing or illegible when filed}$$

### Therefore:

$${P}_{1} = {A}_{0} \frac{N}{{F}_{s}} + {p}_{} ( \sqrt{N} ) \qquad ( 78 )$$

On the other hand:

$${P}_{2} = \frac{1}{{F}_{s}} {\sum }_{k ∈ Ω \text{?}}^{ } {A}_{k}^{f} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} + {η}_{k} ) ) + \frac{1}{{F}_{s}} {\sum }_{k ∈ Ω \text{?}}^{ } {∈}_{k} \\ \text{?} \text{indicates text missing or illegible when filed} \qquad ( 79 )$$

But Σ_{k∈ϵ}ₖ= [symbol] ₚ(δψ\sqrt{l}) and l×N so:

$${P}_{2} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} ) ) + {p}_{} ( \sqrt{N} ) \\ \text{?} \text{indicates text missing or illegible when filed} \qquad ( 80 )$$

Using the same argument as the one detailed above we get:

$${P}_{2} = \frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} ) ) + {p}_{} ( \sqrt{N} ) \\ \text{?} \text{indicates text missing or illegible when filed} \qquad ( 81 )$$

### Additionally:

$$\frac{{A}_{0}}{{F}_{s}} {\sum }_{k ∈ Ω \text{?}}^{ } ( 1 + {K}_{τ}^{mod} cos ( \frac{{ω}_{s} k}{{F}_{s}} - {ϕ}_{τ}^{mod} ) ) = \frac{{A}_{0}}{δψ} {\int }_{\text{?}}^{\text{?}} {\int }_{\text{?}}^{\text{?}} ( 1 + {K}_{τ}^{mod} cos ( {ω}_{s} t - {ϕ}_{τ}^{mod} ) ) {δ}_{Ω \text{?}} dtd ψ + γ ( {ω}_{s} / {F}_{s} ) \\ \text{?} \text{indicates text missing or illegible when filed} \qquad ( 82 )$$

Where γ is a function such that

$$γ ( x ) {\to }_{x \to 0} 0.$$

### Since

$${\int }_{\text{?}}^{\text{?}} {\int }_{\text{?}}^{\text{?}} ( 1 + {K}_{τ}^{mod} cos ( \text{?} - {ϕ}_{τ}^{mod} ) ) {δ}_{Ω \text{?}} dtd ψ + γ ( {ω}_{s} / {F}_{s} ) = \frac{L}{{ω}_{s}} \times {\int }_{\text{?}}^{\text{?}} ( 1 + {K}_{τ}^{mod} cos ( \text{?} - {ϕ}_{τ}^{mod} ) ) d {ϕ}^{′} + ( 1 ) = \frac{L δψ}{{ω}_{s}} ( 1 + \frac{sin ( δψ \text{/} 2}{δψ \text{/} 2} {K}_{τ}^{mod} cos ( - {ϕ}_{τ}^{mod} ) ) + ( 1 ) \\ \text{?} \text{indicates text missing or illegible when filed} \qquad ( 83 )$$

### For

$$\frac{{ω}_{s}}{{F}_{s}}$$

“sufficiently small”, we can write:

$${P}_{2} = \frac{{A}_{0} l}{{ω}_{s}} ( 1 + \frac{sin ( δψ \text{/} 2 )}{δψ \text{/} 2} {K}_{T}^{mod} cos ( - {ϕ}_{T}^{mod} ) ) + ( \sqrt{N} ) \qquad ( 84 )$$

### Finally:

$$P A {M}_{( T , ψ )} = \frac{1}{2 π} \frac{( \frac{1 + sin ( δψ \text{/} 2 )}{δψ \text{/} 2} {K}_{T}^{mod} cos ( ψ - {ϕ}_{T}^{mod} ) + {p}_{} ( 1 \text{/} \sqrt{N} ) )}{1 + {p}_{} ( 1 \text{/} \sqrt{N} )} = \frac{1}{2 π} ( 1 + \frac{sin ( δψ \text{/} 2 )}{δψ \text{/} 2} {K}_{T}^{mod} cos ( ψ - {ϕ}_{T}^{mod} ) ) + {p}_{} ( 1 \text{/} \sqrt{N} ) \qquad ( 85 )$$

### Simulations

We tested our SSCFA and dSSCFA methods on simulated datasets generated by different models. We constructed each simulated signal by combining unit variance Gaussian noise, a slow oscillation centered at fₛ=1 Hz (unless stated otherwise), and a modulated fast oscillation centered at f_{f}=10 Hz (unless stated otherwise). It is important to note that we chose to generate these simulated signals using a method or “model class” that was different from the state space oscillator model we use to analyze the data. For standard processing, significance was assessed with 200 random permutations, fₛ and f_{f} were assumed to be known, and components were extracted with bandpass filters with pass bands set to 0.1-1 Hz for the slow component and 8-12 Hz for the fast component.

Simulating the Slow Oscillation

Neural oscillations are not perfect sinusoids and instead have a broad band character. We simulated a broad band slow oscillation by convolving (filtering) independent identically distributed Gaussian noise with the following impulse response function:

c(t)=c₀(t)cos(ωₛt) (86)

where ωₛ=2πfₛ, c₀ is a Blackman window of order

$$2 ⌊ \frac{1.65 {f}_{s}}{Δ {f}_{s}^{gen}} ⌋ + 1.$$

The smaller the slow frequency bandwidth Δfₛ^{gen}, the closer the signal is to a sinusoid. When necessary, we additionally use a π/2 phase-shifted filter: \tilde{c}(t)=c₀(t)sin(ωₛt) to model an analytic slow oscillation xₜ^{s} from which we deduce the phase ϕₜ^{s}. The resulting series is finally normalized such that its standard deviation is set to σₛ.

To assess the temporal resolution of our method and the standard method, we generated simulated data sets with different rates of time-varying modulation. First, to construct the modulated fast oscillation, we constructed a fast oscillation centered at ω_{f}=2πf_{f} and normalized to σ_{f} as described above and modulated it by:

mₜ=1+Kₜ^{mod} cos(ϕₜ^{s}−ϕₜ^{mod}) (87)

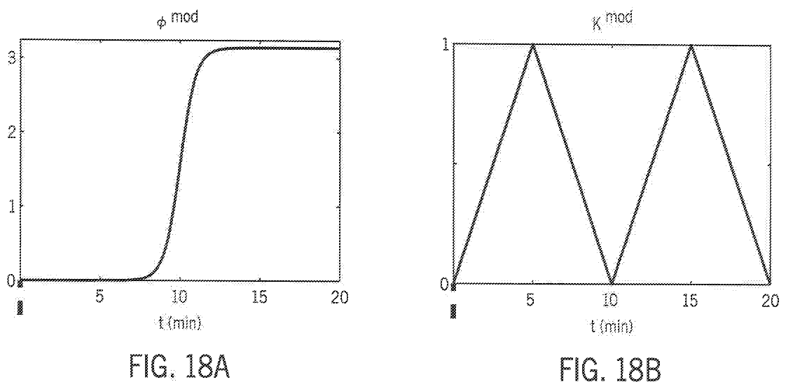

Here, Kₜ^{mod} and phase ϕₜ^{mod} are time varying and can follow dynamics shown by FIGS. 18A and 18B respectively. Simulated data can also be generated using the following alternative modulation function:

mₜ=(1+exp(−λxₜ^{s}u_{ϕ}_{^{mod}}^{T}))⁻¹ (88)

where u_{ϕ}_{^{mod}}^{T}=[cos(ϕ^{mod})−sin(ϕ^{mod})].

Signals with abrupt or sharp transitions can lead to artefactual phase-amplitude cross correlation or phase-amplitude modulation. To assess the robustness of our state-space PAC method under such conditions, we used a Van Der Pol oscillator to generate a signal with abrupt changes. Here, the oscillation x is governed by the differential equation:

$$\frac{{dx}^{2}}{{dt}^{2}} - ∈ {ω}_{0} ( 1 - {x}^{2} ) \frac{dx}{dt} + {ω}_{0}^{2} x = 0 \qquad ( 89 )$$

Equation (89) was solved using an Euler method with fixed time steps.

In summary, the first stage of our algorithm can extract nonlinearities stemming from the modulation before fitting them with a regression model in the second stage. The main consequence of this approach is to inflate the variance in the fast component estimation. See for example the wide CI in the fast oscillation estimate in FIG. 12G. In turn, we resample the fast oscillation amplitudes from a wider distribution than is actually the case. Although this does not affect the estimates of ϕ^{mod}, it does produce a conservative estimate when resampling K^{mod}, i.e., the credible intervals are wider than they might be otherwise. Even so, we find that our approach still performs better than previous methods.

We have presented a novel method that integrates a state space model of oscillations with a para-metric formulation of phase amplitude coupling (PAC). Under this state space model we represent each oscillation as an analytic signal to directly estimate the phase or amplitude. We then characterize the PAC relationship using a parametric model with a constrained linear regression. The regression coefficients, which efficiently represent the coupling relationship in only a few parameters, can be incorporated into a second state space model to track time-varying changes in the PAC. We demonstrated the efficiency of this method by analyzing neural time series data from a number of applications, and illustrated its improved statistical efficiency compared to standard techniques using simulation studies based on different generative models. Finally, we showed how our method is robust to many of the limitations associated with standard cross-frequency coupling analysis methods.

The efficiency of our method stems from a number of factors. First, the state-space analytic signal model provides direct access to the phase and amplitude of the oscillations being analyzed. This linear model also has the remarkable ability to extract a nonlinear feature (the modulation) by imposing “soft” frequency band limits which are estimated from the data. The oscillation decom-position is thus well-suited to analyze physiological signals that are not confined to strict band limits. We also proposed a harmonic extension that can represent nonlinear oscillations, making it possible to better differentiate between true and spurious PAC resulting from bandpass filtering artifacts. The parametric representation of the coupling relation-ship can accommodate different modulation shapes and increases the model efficiency even further.

Overall, we addressed a majority of the significant limitations associated with current methods for certain types of cross-frequency analysis such as PAC analysis. The neural time series are processed more efficiently, frequency bands of interest are automatically selected and extracted, and modeled. Contrary to standard methods, we do not need to average PAC-related quantities across time, reducing the amount of contiguous time series data required. Moreover, the posterior distributions of the signals of interest are well-defined under our proposed model. Sampling from them bypasses the need to build surrogate data, which can obscure non-stationary structure in the data and underestimate the false positive rate. Conversely, because SSCFA estimates the modulation parameters' posterior distribution, we report CI and provide information on the statistical significance of our results as well as the strength and direction of the modulation. Our dynamic estimation of PAC hence makes it possible to base inference on much shorter windows—as short as 6 seconds for slow 0.1-1 Hz signals. Other novel models have been proposed to represent PAC, including driven autoregressive models (DAR) and generalized linear models (GLM). As we saw earlier, SSCFA performs better than the DAR and standard approaches, particularly when the signal to noise is low. The GLM represents the modulation relationship parametrically as we do, but in a more general form that may be less efficient statistically, and provides confidence intervals using the bootstrap. Both the DAR and GLM approaches remain reliant on traditional bandpass filtering for signal extraction, and thus remain vulnerable to the crucial problems introduced by these filters. Our method is the first to use state space models combined with a parametric model of the modulation.

Such improvements could significantly improve the analysis of future studies involving CFC, and could enable medical applications requiring near real-time tracking of CFC. One such application could be EEG-based monitoring of anesthesia-induced unconsciousness. During propofol induced anesthesia, frequency bands are not only very well defined, but the PAC signatures strongly discriminates deep unresponsiveness (peakmax) from transition states (through-max), most likely reflecting underlying changes in the polarization level of the thalamus. Thus, PAC could provide a sensitive and physiologically-plausible marker of anesthesia-induced brain states, offering more information than spectral features alone. Accordingly, one study reported cases in which spectral features could not perfectly predict unconsciousness in patients receiving general anesthesia. In this same data, CFC measures (peakmax) could more accurately indicate a fully unconscious state from which patients cannot not be aroused. In the operating room, drugs can be administered rapidly through bolus injection, drug infusion rates can change abruptly, and patients may be aroused by surgical stimuli, leading to corresponding changes in patients' brain states over a time scale of seconds. These rapid transitions in state can blur modulation patterns estimated using conventional methods. Faster and more reliable modulation analysis could therefore lead to tremendous improvement in managing general anesthesia. Conventional methods are impractical since they require minutes of data to produce one estimate; in contrast our method can estimate CFC on a time-scale of seconds that is compatible with such applications.

Since CFC analysis methods were first introduced into neuroscience, there has been a wealth of data suggesting that CFC is a fundamental mechanism for brain coordination in both health and disease. Our method addresses many of the challenging problems encountered with existing techniques, while also significantly improving statistical efficiency and temporal resolution. This improved performance could pave the way for important new discoveries that have been limited by insufficient analysis methods, and could enhance the reliability and efficiency of PAC analysis to enable their use in medical applications.

Methods of performing cross-frequency analysis between an oscillation wave and a range of frequencies are provided below. Although the below methods typically use filtering techniques to isolate a slow frequency and a broadband range of frequencies, it is understood that the state space modeling techniques presented above can be used in place of at least a portion of the filtering techniques in order to better fit an oscillation and potentially provide more accurate estimations of the relationship between an oscillation such as a slow wave and a range of frequencies such as a broadband range of about 5 Hz-50 Hz. How the above state space model and associated fitting techniques can be used to estimate the relationship between the slow wave and the broadband wave will also be explained.

A controversy has arisen over the last several years regarding the role that frontal and posterior cortices play in mediating consciousness. The “posterior hot zone” hypothesis proposes that posterior sensory and sensory association cortices are the principal mediators of consciousness, distinct from prefrontal cortex. In contrast, some groups argue that frontal-posterior inter-actions are critical to ignite a conscious percept. The controversy has recently expanded to include studies of unconsciousness, and how functional impairment in frontal and posterior regions relates to loss of consciousness.

Different states of unconsciousness such as anesthesia, NREM sleep, and coma, have distinct electrophysiological signatures. One feature that is common to all of them, however, is slow wave activity, seen in the electroencephalogram (EEG) as large deflections alternating at approximately 1 Hz. These waves are thought to be large-scale indicators of underlying cortical up- and down-states in which neurons cycle between sustained periods of depolarization (up-states) and hyperpolarization (down-states). In the depolarized up-state, neurons may fire, but in the hyperpolarized down-states, neurons are silent.

While slow wave activity during unconsciousness is spatially widespread, recent studies have examined how the spatial distribution of slow wave dynamics relates to states of unconsciousness. During NREM sleep, slow wave power over posterior areas is stronger during non-dreaming sleep compared to dreaming sleep. In contrast, frontal areas have been implicated in anesthesia-induced unconsciousness. Pharmacological stimulation of prefrontal cortex can restore behavioral arousal in anesthetized animals, alongside an apparent decrease in slow oscillation power. Anesthesia-induced slow oscillations modulate frontal alpha oscillations at different phases (“troughmax” and “peakmax” dynamics) depending on the depth of anesthesia: when frontal alpha power is strongest at the peak of the slow wave (peakmax), it seems to reflect a deep anesthetic state in which subjects cannot be aroused from unconscious-ness even in the presence of painful stimuli. These data suggest that different behavioral states sharing similar slow wave power can be dissociated from each other based on the modulatory influence of the slow wave on higher frequency rhythms. Could this shift in perspective from slow wave power to slow wave modulation be used to reconcile the roles of posterior and frontal cortices in mediating unconsciousness?

While previous cross-frequency modulation analyses during unconsciousness have focused on the influence of the slow wave on the alpha rhythm, the interpretation of slow waves as cortical up- and down-states would suggest that EEG power at all frequencies should be coupled to the slow wave, since up- and down-states affect both rhythmic and non-rhythmic neural activity. Following this idea, we introduce a new analysis method to track the modulatory influence of the slow wave across a broad range of frequencies measured in the EEG. We use this method to analyze slow wave modulation in different cortical regions across different states of propofol-induced unconsciousness. First, we find that frontal peakmax dynamics are a broadband phenomenon, suggesting that peakmax dynamics reflect underlying cortical up-and down-states. Moreover, we find that posterior and frontal regions exhibit this broadband modulation by the slow wave at different states of unconsciousness: posterior regions at lighter levels of unconsciousness and frontal regions at deeper levels of unconsciousness. This result supports the idea that anesthesia-induced unconsciousness is not a single state but rather multiple states, each showing different levels of posterior and frontal cortical disruption alongside corresponding differences in conscious processing and behavioral arousability.

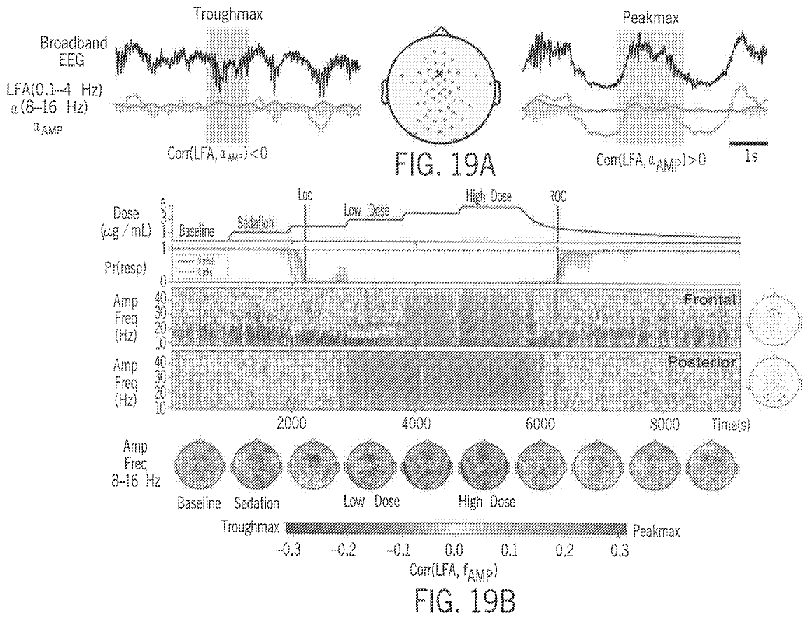

We quantified cross-frequency coupling using a correlation between the band-pass filtered slow voltage (0.1-4 Hz) and the instantaneous amplitude of the band-pass filtered high-frequency signal as shown in FIG. 19A. This correlation provides a one-dimensional summary of cross-frequency coupling: if the correlation is positive, it means that the high frequency amplitude is higher when the slow voltage is positive (peakmax coupling); if the correlation is negative, it means that the high frequency amplitude is higher when the slow voltage is negative (troughmax coupling). We can then vary the frequency band used for the high frequency amplitude, to see how different frequencies relate to the slow wave. Using this approach, we can look at how peakmax and troughmax dynamics vary across electrodes and frequencies during the transition to unconsciousness.

FIG. 19B shows how peakmax and troughmax dynamics play out for a representative subject: the subject was administered propofol in increasing doses every 14 minutes while performing an auditory task with a button-click response. When the propofol dose was sufficiently high, the subject stopped responding to the stimulus (LOC). After 5 increasing levels of propofol, the dose was reduced and eventually the subject started responding to the stimulus again (ROC).

The correlation-based cross-frequency coupling over frontal electrodes reaffirms the basic result from previous work on the alpha rhythm: at 10 Hz, coupling to the slow wave begins as troughmax dynamics, switches to peakmax at higher doses, and transitions back through troughmax during recovery. By looking beyond 10 Hz, however, we can see that the troughmax dynamics begin well before LOC at higher frequencies up to about 20 Hz, frequencies that show higher power during propofol sedation in which subjects remain conscious. This is also consistent with the observation that patients may be conscious during troughmax dynamics.

Perhaps the most striking result in the cross-frequency coupling is that the peakmax pattern extends across all frequencies (5-50 Hz). Since there are no narrow-band rhythms in the signal other than alpha and slow, this result suggests that non-rhythmic broadband activity is coupled to the slow wave. This is consistent with an interpretation in which peakmax dynamics reflect underlying cortical up- and down-states, wherein cortical activity, whether rhythmic or not, is shut down during the trough of the slow wave.

Comparing the cross-frequency coupling between the frontal and posterior electrodes, we can see that a broadband peakmax pattern develops in posterior channels shortly after loss of consciousness, at the same time as frontal troughmax. That is, even before frontal channels transition into peakmax dynamics, posterior channels already show broadband coupling to the slow wave. In all subjects, the onset of posterior peakmax dynamics occurred after LOC and before the onset of frontal peakmax dynamics. This result is further shown in the topographic maps of the 8-16 Hz cross-frequency coupling by level: shortly after loss of consciousness, a collection of frontal electrodes still have troughmax coupling while electrodes over posterior electrodes show peakmax coupling. As the propofol level is increased, the frontal electrodes begin to participate in the peakmax coupling. This suggests that broadband silencing of cortical activity by the slow wave begins first over posterior areas before extending frontally at higher doses of propofol.

For the rest of the analyses, we identified four levels of interest: (1) Baseline, before the administration of propofol; (2) Sedation, the level before the subject stopped responding; (3) Unconscious Low Dose, the first level after the subject stopped responding; and (4) Unconscious High Dose, the highest level of propofol that did not result in burst suppression, a coma-like state beyond what is required for unconsciousness. This allowed us to combine information across subjects, given that the timecourse of LOC and ROC were different for each subject.

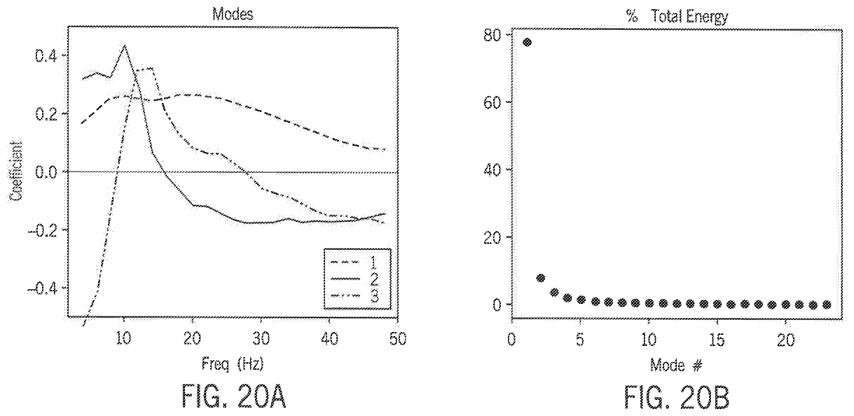

Since the patterns of cross-frequency coupling across frequency varied by electrode, propofol level, and subject, we wanted to identify the coupling patterns that best summarized this activity. To do this, we used a non-centered principal component analysis (PCA) to decompose the cross-frequency coupling patterns into principal frequency modes as shown in FIG. 20A. The principal modes are orthogonal patterns across frequency that capture successively smaller proportions of the total energy in the matrix representing cross frequency coupling by frequency, electrode, propofol level, and subject.

The first principal mode is positive for all frequencies, so it represents broadband effects in the cross-frequency coupling, and it captures 78% of the total energy. The second and third principal modes, which have narrow-band peaks in the alpha and beta frequency ranges, respectively, capture much less of the energy. Hence the entrainment of broadband cortical activity to the slow wave is by far the biggest contributor to the cross-frequency coupling during propofol anesthesia. The percent of total energy of each mode is shown in FIG. 20B.

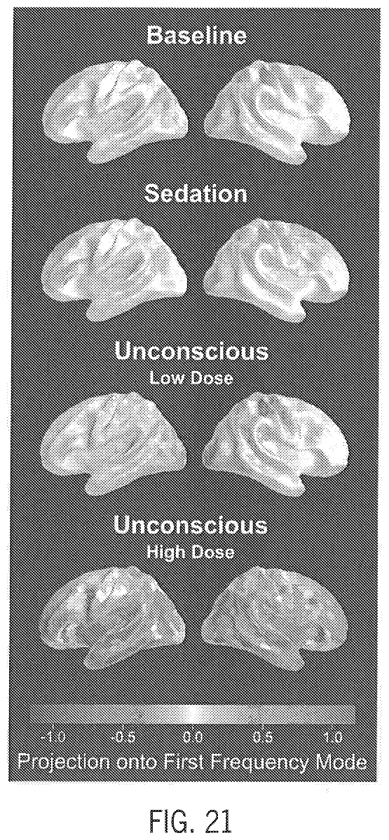

This dominant first frequency mode is of particular interest, because it can capture the broadband peakmax pattern observed in the individual subject summaries. In particular, we would like to use the first principal mode to describe the spatial distribution of the broadband peakmax phenomenon among the cortical generators of the EEG during progressively higher doses of propofol. In order to do so, we performed the cross-frequency coupling analysis on the source-localized EEG signals, and projected the resulting cross-frequency coupling patterns across cortical location onto the first principal mode from the sensor-space analysis described above. FIG. 21 shows the resulting projections, morphed to the Freesurfer-average surface and averaged across subjects. On these surfaces, a positive projection (red) represents broadband peakmax coupling to the slow wave.

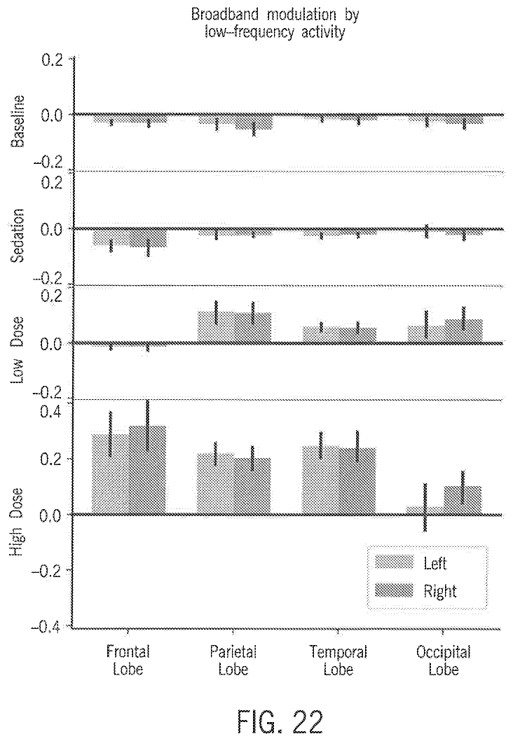

Consistent with the individual subject sensor-space results, broadband peakmax coupling to the slow wave is present over posterior cortical areas during the Unconscious Low Dose condition. It strengthens and spreads to frontal cortical areas during the Unconscious High Dose condition. In FIG. 22, a graph of broadband frequency modulation by low frequency activity at different locations of the brain during different states, we can see that during the Unconscious Low Dose condition the parietal, temporal, and occipital lobes all have broadband peakmax coupling, but the frontal lobe has virtually no broadband coupling to the slow wave. During the Unconscious High Dose condition, in contrast, the frontal lobe joins the other lobes in broadband peakmax coupling.

Together these results suggest that broadband peakmax coupling to the slow wave is a major contributor to cross-frequency coupling dynamics during propofol anesthesia, as can be seen in FIG. 20. Furthermore, posterior cortical areas are the first to exhibit broadband peakmax coupling after loss of consciousness, followed by frontal areas at high doses of propofol, as can be seen in FIGS. 21 and 22.

We propose that the broadband coupling to the slow wave that we see in the EEG signal is a macro-scale indicator of regional cortical up- and down-states. Population spiking activity has a broadband effect on the neural power spectrum, so the entrainment of population spiking activity to the slow wave during cortical up- and down-states should cause broadband power to be strongest at a particular phase of the slow wave. This idea is supported by micro-scale evidence in electrocorticography and local field potential data showing that up-states are associated with simultaneous increases in both population spiking activity and broadband power (<50 Hz). While it is common to assume that increased slow power in the EEG always corresponds to up- and down-states in cortical firing, our data suggests that the slow wave must reach a critical amplitude, which may vary by location, before it entrains the populationspiking activity enough to produce broadband modulation. Posterior areas cross this threshold at lower doses of propofol than frontal areas. The idea of a critical threshold for the slow wave is consistent with the slow wave activity saturation hypothesis, in which saturation of the slow wave power accompanies a disconnection between thalamus and sensory cortex. We hypothesize that the cortical up- and down-states themselves provide a mechanism for this sensory isolation. Under this interpretation, broadband peakmax dynamics signify that local cortical activity is being disrupted by up- and down-states on a scale large enough to be detected in the EEG.

The ability to monitor a patient's state of unconsciousness during general anesthesia has obvious practical benefits, but identifying principled methods for doing so remains an open question. We can make a distinction between unconscious states in which patients can be aroused to consciousness by external stimuli (arousable), versus those in which patients cannot be aroused (unarousable). Some recent data suggest indirectly that patients can be arousable when frontal alpha rhythms are strongest at the trough of the slow wave (troughmax), but are unarousable when the frontal alpha rhythms are strongest at the peak of the slow wave (peakmax). Here we show that when frontal alpha rhythms are coupled to the peak of the slow wave (peakmax), broadband spectral power over both frontal and posterior cortices, is also coupled to the slow wave. Hence unarousability appears to be associated with broadband coupling to the slow wave over both frontal and posterior cortex. At lower anesthetic doses in which patients are unconscious but potentially arousable, we find that the posterior cortex engages in broadband peakmax dynamics while the prefrontal cortex participates in (alpha-band) troughmax dynamics. Thus, these different spatial patterns of cross-frequency coupling appear to indicate different states of unconsciousness. Knowledge of these states could be used by anesthesiologists to establish different arousable or unarousable states of unconsciousness appropriate to the clinical circumstances.

The “posterior hot zone” and fronto-posterior hypotheses propose starkly different roles for posterior and frontal circuits in mediating consciousness. Anesthesia introduces another dimension to this problem in that unconscious patients may or may not be arousable by external stimuli depending on the drug dose. Along this dimension, the prefrontal cortex appears to play a central role, in which stimulation of prefrontal cortex can jump-start consciousness. Our results would seem to harmonize these viewpoints, in that the disruption of frontal versus posterior cortices coincides with different states of unconsciousness. First, both the posterior hot zone and fronto-posterior hypotheses predict that disruption of posterior areas should degrade the contents of consciousness. In our results, posterior areas are the first to show broadband peakmax at low doses of propofol, when subjects are unconscious but may be arousable. These posterior areas appear strikingly similar to those with higher slow-wave power during non-dreaming sleep. This is consistent with the interpretation that posterior cortical regions mediate the contents of consciousness, which are disrupted by alternating up- and down-states during unconsciousness. Second, frontal areas develop broadband peakmax coupling at higher doses of propofol, in a state associated with unarousability. This is consistent with the interpretation that prefrontal cortex is required for behavioral arousal to consciousness and that alternating up- and down-states in prefrontal cortex prevent this arousal.

The main result of this study is that brain-wide cross-frequency coupling analysis captures reliable but spatially-varying changes in brain dynamics during the transition to unconsciousness and unarousability under propofol anesthesia. We interpret broadband coupling to the peak of the slow wave to be a macro-scale indicator of up- and down-states in the micro-scale cortical activity that serve to disrupt cortical function. This functional disruption encompasses both frontal and posterior cortical areas at different states of unconsciousness. Our results suggest that unconsciousness under anesthesia is not a singular phenomenon but rather involves several distinct shifts in brain state that disrupt posterior cortical processing of the “contents” of consciousness distinct from prefrontal arousal and awareness of those contents.

A study was performed to analyze data that had been previously described. Ten healthy volunteers between the ages of 18 and 36 participated. Prior to the study, structural MRI scans were obtained for each subject (Siemens Trio 3 Tesla, T1-weighted magnetization-prepared rapid gradient echo, 1.3-mm slice thickness, 1.3 1 mm in-plane resolution, TR/TE=2530/3.3 ms, 7 flip angle). Cortical reconstruction was performed with the FreeSurfer image analysis suite.

After at least 14 minutes of eyes-closed rest, subjects were administered propofol anesthesia, increasing propofol dosage every 14 minutes to target 1, 2, 3, 4, or 5 μgmL⁻¹ effect-site concentration. During emergence, propofol dose was decreased in three 14-minute steps targeting effect-site concentrations of 0.5 μgmL⁻¹, 1.0 μgmL⁻¹, and 1.5 μgmL⁻¹ less than the dose at which the subject stopped responding to the auditory cues. Electroencephalogram (EEG) recordings were collected with a high density (64 channel) BrainVision MRI Plus system (Brain Products) with a sampling rate of 5,000 samples per second and the electrode positions were digitized (Polhemus FASTRACK 3D).

Throughout the study, subjects performed a task in which they responded to an auditory click or word with a finger-press of a button. Loss of consciousness (LOC) was defined when a state-space model estimated a less than 5% probability of a correct response and remained so for at least 5 minutes. Similarly, return of consciousness (ROC) was defined as the first time during emergence when the state-space model reached a 5% probability of a correct response and remained so for at least 5 minutes.

EEG preprocessing: Channels with severe, persistent artifacts were identified by visual inspection and removed from the analysis. We also removed electrodes that showed signs of being electrically bridged according to the eBridge software package.

The cross-frequency coupling analysis described below uses versions of the signal that have been bandpass filtered in the slow band (0.1-4 Hz) and a set of higher bands between 4 and 50 Hz, each 2 Hz in bandwidth with a 1 Hz transition band. We filtered the data for the entire session into these bands using a zero-phase FIR filter and downsampled to 200 samples per second. We chose the upper limit of the higher bands, 50 Hz, in order to avoid line noise and capture the frequencies with the largest EEG power.

For sensor-level analyses, we applied a Laplacian reference based on first nearest neighbors after filtering.

Because of the stepped structure of the propofol dosage, subjects had approximately 12 minutes at each dosage with constant predicted effect-site concentration. We call these periods “levels”, and defined four levels of interest: Baseline, Sedation, Unconscious Low Dose, and Unconscious High Dose. Baseline was defined as the period before the onset of propofol, Sedation was defined as the level prior to LOC, Unconscious Low Dose was defined as the first full level after LOC, and Unconscious High Dose was defined as the highest level of propofol or the level before burst suppression (for subjects who entered burst suppression). For analyses performed on these levels, we selected 10 artifact-free epochs 30 s long from each level for each subject. Two of the subjects stopped responding during the first level of propofol, so they did not have a sedation period. As a result, figures summarizing across subjects represent 10 subjects for Baseline, Low Dose, and High Dose, and 8 subjects for Sedation.

Cross-frequency coupling analysis: Under propofol, high frequency amplitude is much more likely to couple to the peak or the trough of the slow wave than to the rising or falling phases. Hence we summarize cross-frequency coupling using a metric that is either positive (peakmax: high frequency amplitude is higher during the peak of the slow wave) or negative (troughmax: high frequency amplitude is higher during the trough of the slow wave). In particular, we use the Pearson correlation coefficient between the instantaneous amplitude of the band-passed high frequency signal and the band-passed slow voltage (0.1-4 Hz):

$$Corr ( V , A ) = \frac{{V}^{T} A}{\sqrt{{V}^{T} V} \sqrt{{A}^{T} A}} \qquad ( 90 )$$

Where V is is a column vector representing a timeseries of the slow voltage and A is a vector representing the corresponding timeseries of the instantaneous amplitude of the high frequency band. The instantaneous amplitude was computed using the magnitude of the analytic signal of the bandpass filtered signal. Before computing the correlation, the instantaneous amplitude for each 30 s epoch was centered by subtracting the mean. V was not explicitly centered because its expected value is zero.

In order to find the average correlation across space (electrodes, source locations) and/or time (multiple 30 s epochs), the dimensions to be averaged are stacked vertically in the V and A column vectors in Equation 1. Since the correlation represents the covariance between the signals (in the numerator, V^{T}A) normalized by the standard deviations of each signal (in the denominator, \sqrt{V^{T}V} and \sqrt{A^{T}A}, stacking the signals vertically has the effect of estimating the covariance and standard deviations separately before performing the normalization.

Note that this analysis does not involve estimating the phase of the slow wave, a typical step in phase-amplitude coupling analysis. We chose not to estimate phase for three reasons. First, phase-amplitude coupling in propofol clusters around zero (slow wave peak, when the signal is positive) and 1T (slow wave trough, where the signal is negative). Second, the slow wave is thought to reflect up states and down states, regardless of whether it is well-described by a sinusoid. Early in propofol-induced unconsciousness, the slow wave is often quite irregular in waveform shape and frequency. We chose to use a wider band for the slow wave, 0.1-4 Hz, in order to capture more of the non-sinusoidal features of the waveform. Under propofol anesthesia, there is no narrow-band delta frequency rhythmic activity (1-4 Hz), so the contribution of this frequency range to the band-passed signal was non-rhythmic, affecting the waveform shape. By using correlation to describe the relationship of the high frequency amplitude to the non-sinusoidal low frequency waveform, we are therefore better able to capture the relationship between high frequency activity and the up and down fluctuations that are apparent in the broadband signal (FIG. 19A). Finally, reducing the problem to one dimension (positive and negative coupling) simplified the analysis of a single location and frequency, allowing us to expand the analysis to other frequencies and locations.

FIG. 19A shows the computation for a frontal electrode, computing the correlation between the slow voltage (low-frequency activity, LFA) and the alpha band (a, 9-11 Hz) during two time windows. For the time window on the left, the alpha amplitude is higher when the slow voltage is negative, leading to a negative correlation (troughmax). For the time window on the right, the alpha amplitude is higher when the slow voltage is positive, leading to a positive correlation (peakmax). We can perform this analysis for all 30 s epochs in the session and for a range of amplitude frequencies (2 Hz bands between 4 and 50 Hz): this results in a modulogram showing the cross-frequency coupling between each frequency and the slow wave across time in the session for the selected electrode. Similarly, we can compute the correlations for each electrode within propofol levels, and show the spatial distribution of cross-frequency coupling between the alpha band and the slow wave as a function of propofol level.

We performed the cross-frequency coupling analysis across amplitude bands, time, electrodes, and subjects. We characterize time in two ways. The first was a session-based analysis, in which the coupling was assessed for every 30 s epoch in the session separately, resulting in modulograms such as the frontal and posterior summaries in FIG. 19B: these modulograms represent the average correlations across several electrodes, indicated in the scalp maps to the right. The second way to characterize time was based on propofol level, in which the coupling was assessed over 10 artifact-free 30 s epochs at each propofol level. Hence, each coupling value represents the correlation over 300 seconds of data, and we have one such value for each electrode, level, and subject. This level-based analysis was used for the topoplots in FIG. 19B and for the Principal Component Analysis described below.

Source localization Source localization was performed by minimum norm estimation using the MNE-python toolbox (27.martinos.org/mne/). The noise covariance was assumed to be diagonal, estimated from the EEG using the spectral power above 65 Hz. The source space was an ico-3 decimation of the Freesurfer reconstruction of the cortical surface and the sources were constrained to be perpendicular to the cortical surface. The forward model was computed using a 3-layer boundary element model, eliminating any sources within 5 mm of the inner skull surface.

To perform the cross-frequency coupling analysis in source space, the band-passed signals in the slow band and each 2 Hz band between 4 and 50 Hz were source localized using the methods described above. For the cortical surface representations (FIG. 21), the cross-frequency coupling analysis described above was applied to each source location for each subject, for the four levels of interest. The average cross-frequency coupling within lobes (FIG. 22) used the same technique, but the signals for all of the sources in a lobe were combined: the 10 epochs (for each level) for all of the sources in the lobe were stacked vertically in the V and A column vectors in Equation 1.

Non-centered Principal Component Analysis and Projection into Source Space: To identify patterns of coupling across frequency that explained most of the observed features, we performed a non-centered principal component analysis on the sensor-space coupling patterns across propofol levels and subjects. The level-based coupling results for the four levels of interest (baseline, sedation, unconscious low dose, and unconscious high dose) were combined into an aggregate matrix A(f, i) where f indexes the amplitude band and i indexes the propofol level, electrode, and subject (i.e. the rows contain all of the levels, electrodes, and subjects stacked). Cross-frequency coupling results for two electrodes during the four levels of interest, for all subjects were then generated: the goal of the analysis was to decompose these patterns into components that capture their major features across frequencies. Principal component analysis involves a singular value decomposition:

A=USV^{T} (91)

In the decomposition, U is a matrix whose columns are the principal modes. The first column is the first principal mode, meaning the pattern across frequencies that captures the most energy in the A matrix. The second column is the second principal mode, which captures the most energy in the data after the first mode has been removed. The principal modes are orthogonal, and they all have unit length. In order to preserve the mapping of positive values to peakmax and negative values to troughmax, the principal modes were flipped (multiplied by −1) as necessary so that the largest element would be positive. The S matrix is diagonal, and can be used to estimate the percent of the total energy that is explained by the i^{th} mode:

$$Percent Energy ( j ) = \frac{{S ( j , j )}^{2}}{{\sum }_{k}^{F} {S ( k , k )}^{2}} \qquad ( 92 )$$

Note that we chose to use non-centered PCA, which decomposes the total energy, rather than centered PCA, which decomposes variance. Non-centered PCA is particularly useful in situations where the origin has special significance that would be lost if the data were centered by subtracting the mean. For patterns of cross frequency coupling, the origin represents a situation in which the EEG signal contains no coupling to the slow wave at any frequency. In contrast, the mean represents the average coupling to the slow wave over subjects, propofol levels, and electrodes (as a function of amplitude frequency). We feel that the origin has more significance than the mean, and as a result the non-centered PCA is more interpretable than the more common centered PCA.

To quantify how the principal modes were represented in source space, we projected the source-space patterns for each subject onto the first sensor-space principal mode. First, the coupling results from the source-space analysis were combined into an aggregate A^{source}(f, i) matrix, where i indexes the propofol level, source location (or lobe), and subject. Then the projection of A^{source} onto U is:

P=U^{T}A^{source} (93)

The first row of the resulting P matrix contains the projection of source-space coupling patterns (for each propofol level, source location, and subject) onto the first principal mode. Since the first principal mode is relatively constant across frequencies (see FIG. 20), the projections onto this mode represent cross-frequency coupling that is broadband. Also, since the first principal mode is positive for all frequencies, positive projections reflect broadband peakmax coupling (all frequencies coupled to the peak of the slow wave), and negative projections reflect broad-band troughmax coupling (all frequencies coupled to the trough of the slow wave).

The projections of the (subject×level×lobe) coupling patterns onto the first principal mode were averaged across subjects to yield the estimates shown in FIG. 22, representing the average contribution of the first mode to the cross-frequency coupling in that lobe. The 95% confidence intervals were obtained using a bootstrap over subjects.

To summarize the results across subjects on the entire cortical surface, we morphed the projections of the (subject×level×sourcelocation) coupling patterns onto the first principal mode to the Freesurfer-average surface using MNE-python, and we averaged the resulting maps across subjects. The resulting maps estimate the contribution of the chosen mode to the cross frequency coupling at each cortical location (see FIG. 21).

As mentioned above, the state space model can be used in detecting cross-frequency relationships between a slow component and a range of broadband frequencies. In order to use the state space model in a state space model-based cross-frequency coupling analysis method, the following parameter substitutions can be made to equations (3) and (4) can be substituted as shown below to only obtain a slow component. Equations (3) and (4) are repeated for reference.

yₜ=Mxₜ+vₜ, vₜ˜ [symbol] (0, R) (3)

xₜ=Φxₜ₋₁+uₜ, uₜ˜ [symbol] (0, Q) (4)

Where M=[1 0], xₜ=xₜ^{s}, Q=Qₛ, Φ=aₛR(ωₛ)

The model can then be fit using an Expectation-Maximization algorithm as described above. Then, the two elements of the vector timeseries xₛ^{t} (the real and imaginary parts, respectively) can be used as the band-pass filtered slow voltage (i.e. the slow signal) in the cross-frequency coupling estimation method described above. The amplitude in the high frequency bands can still be computed using band-pass filtering and taking the magnitude of the analytic signal (i.e. the Hilbert transform method).

The cross-frequency coupling can then be quantified using equation (90), which is repeated for reference:

$$Corr ( V , A ) = \frac{{V}^{T} A}{\sqrt{{V}^{T} V} \sqrt{{A}^{T} A}} \qquad ( 90 )$$

where V is a column vector containing either the first or second element of xₜ^{s} and A is a vector representing the corresponding timeseries of the instantaneous amplitude of the high frequency band. Before computing the correlation, the instantaneous amplitude should be centered by subtracting the mean. V does not have to be explicitly centered because its expected value is zero.

A graph resembling FIG. 19B can then be generated for each element of xₜ^{s}. The analysis corresponding to the first element (i.e. the real part) of xₜ^{s} will capture coupling of the high frequency activity to the peak (postive, peakmax) and trough (negative, troughmax) of the slow wave, and the patterns should be qualitatively very similar to the original analysis across time, frequency, and electrode location. The analysis corresponding to the second element (i.e. the imaginary part) of xₜ^{s} will capture coupling of the high frequency activity to the falling phase and the rising phase of the slow wave. In some embodiments, residuals remaining after fitting the slow oscillation can be bandpassed and used in place of the bandpassed original EEG signal. Using residuals may remove any high-frequency elements of the estimated slow wave from the broadband signal.

One potential issue with the state space model-based cross-frequency coupling analysis method described above is that if the signal contains strong oscillations or localized spectral power outside of the slow range, the EM algorithm could perform undesirably: e.g. it could shift the frequency fₛ outside of the range associated with slow waves (less than about 1 Hz), and/or it could increase the variance σₛ² to capture both the high frequency power and the slow wave.

On potential solution to the above problem is to choose the number of oscillatory components for the state-space model and use the resulting hidden states, one of which should correspond to the slow wave, and the rest will correspond to higher frequency oscillations. The correlation between the instantaneous amplitude of the high frequency components and the real/imaginary parts of the slow component can then be computed. The residuals of the model (reflecting “non-oscillatory” components of the signal) can then be determined, and the Hilbert transform method can be performed to get the instantaneous amplitude of the high frequency residuals (in a set of frequency bands, as described above), and then the correlation between these bands and the real/imaginary parts of the slow component can be calculated.

Another potential solution is instead of using the Hilbert transform method to compute the instantaneous amplitude of the high frequency signals, we could get the instantaneous amplitude from the state space model using a set of high frequency components that tile the higher frequencies. In other words, the canonical state space equations (3,4) can be fit with components for the slow wave xₜ^{s} as well as a set of high frequency hidden states xᵢ^{j}, where i indexes frequencies (e.g. between 5 and 50 Hz). The EM algorithm may behave unpredictably in this case, since there may not generally exist a narrow-band oscillation for every high-frequency component. So to allow this method to work, a user would have to choose the frequencies fᵢ (location of the peak of the frequency band) and the autoregressive coefficients aⱼ (sharpness of the peak) for the high frequency bands a priori, without updating them in the EM algorithm.

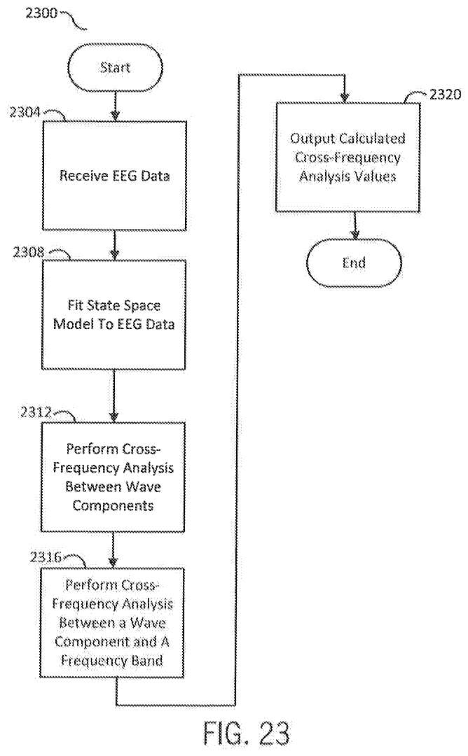

Referring to FIGS. 2 and 11 as well as FIG. 23, an exemplary process 2300 for performing cross-frequency analysis using fitted state space models is shown. The process 2300 can be implemented as instructions on at least one memory. The instructions can then be executed by at least one processor.

At 2304, the process 2300 can receive EEG data. The EEG data can be received from a patient monitoring device such as patient monitoring device 202 described above in conjunction with FIG. 2. The EEG data can be interpreted as a waveform. The process 2300 can then proceed to 2308.

At 2308, the process 2300 can fit a state space model to the EEG data. 2308 can include at least a subset of the steps of process 1100, for example 1108-1128. The process 2300 can then use the fitted model to perform cross-frequency analysis in subsequent steps. The process 2300 can then proceed to 2312.

At 2312, the process 2300 can perform cross-frequency analysis on a slow wave component and an alpha wave component of the fitted model. The process 2300 can calculate one or more parametric representations of phase amplitude coupling between two wave components. The process 2300 can calculate SSCFA using equation (33). Alternatively or in addition, the process 2300 can calculate dSSCFA using equation (49). Both SSCFA and dSSCFA can provide an accurate estimate of phase amplitude coupling between the alpha wave component and the slow wave component, and more specifically how the phase of the slow wave component modulates the amplitude of the alpha wave component, without relying on time binning. As mentioned above, the dSSCFA imposes a temporal continuity constraint on modulation parameters across time windows of the EEG data. In other words, dSSCFA is calculated based on a temporal constraint. The parametric representation(s) PAC provided by SSCFA and dSSCFA provide an indication of peakmax and/or through-max coupling between the slow wave omponent and the fast wave component. The process 2300 can then proceed to 2316.

At 2316, process 2300 can perform cross-frequency analysis between the slow wave component and a range of frequencies of the EEG data. For example, the relationship between the slow wave component and the frequencies 5 Hz-50 Hz may be estimated. Correlation between the slow wave component and the range of frequencies can be calculated using equation (90). In equation (90), V is a column vector containing either the first or second element of xₜ^{s}, which is obtained from the fitted model. A is a vector representing the corresponding timeseries of the instantaneous amplitude of a high frequency band (i.e. 5 Hz-50 Hz). A can be determined by applying a bandpass filter configured to pass the high frequency band to the EEG data. In some embodiments, residuals remaining after fitting the slow wave component to the EEG data can be bandpassed using a bandpass filter configured to pass the high frequency band. Using the residuals to determine A may remove any high-frequency elements of the estimated slow wave from the broadband signal, potentially providing a better signal. The process 2300 can then estimate the correlation between the slow wave and the high frequency band using equation (90). The process 2300 can then proceed to 2320.

At 2320, the process can output the SSCFA, the dSSCFA, and or the correlation between the slow wave and the high frequency band model to a display for viewing by a user and/or to a memory for storage and/or use by another process. Other processes may further process calculated cross-frequency analysis values (i.e. the SSCFA, the dSSCFA, and or the correlation between slow wave and high frequency band) to generate a report in order to make the values more easily understood by a human user, such as mapping a plurality of SSCFA values calculated from a plurality of electrode readings to a model of a brain based on a location of each electrode. The report can also be output to a display for viewing by a user and/or to a memory for storage and/or use by another process. The process 2300 can then end.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A method for estimating at least one oscillatory component of a patient brain state, the method comprising:
receiving electroencephalogram (EEG) data;
fitting a space state model to the EEG data, the state space model comprising at least one oscillatory component;
estimating at least one value of cross-frequency coupling of the EEG data based on the state space model;
generating a report based on the at least one value of cross-frequency coupling; and
outputting the report to at least one of a display and a memory.

2. The method of claim 1, wherein the at least one value of cross-frequency coupling comprises an estimated value of phase amplitude modulation between a first wave component and a second wave component included on the state space model.

3. The method of claim 8, wherein the first wave is a slow wave component and the second wave is an alpha wave component.

4. The method of claim 3, wherein the estimated value of phase amplitude modulation is calculated based on a phase of the slow wave component and an amplitude of the alpha wave component.

5. The method of claim 1, wherein the at least one value of cross-frequency coupling comprises a correlation value between an oscillatory component included in the state space model and a band of frequencies of the EEG data.

6. The method of claim 1, wherein the fitting the state space model comprises fitting harmonic frequencies of each oscillatory component included in the at least one oscillatory component.

7. The method of claim 1, wherein the at least one oscillatory component includes an alpha component, and the alpha component is associated with prior distribution for a center frequency.

8. The method of claim 7, wherein the prior distribution is a Von Mises prior.

9. The method of claim 1, wherein a damping factor of the state space model is constrained with a prior distribution.

10. The method of claim 9, wherein the prior distribution is a beta distribution.

11. A system for estimating at least one oscillatory component of a patient brain state, the system comprising:
a plurality of sensors configured to receive electroencephalogram (EEG) data;
a processor configured to receive the EEG data and carry out steps including:
fitting a space state model to the EEG data, the state space model comprising at least one oscillatory component;
estimating at least one value of cross-frequency coupling of the EEG data based on the state space model;
generating a report based on the at least one value of cross-frequency coupling; and
a display configured to display the report.

12. The system of claim 11, wherein the at least one value of cross-frequency coupling comprises an estimated value of phase amplitude modulation between a first wave component and a second wave component included on the state space model.

13. The system of claim 18, wherein the first wave is a slow wave component and the second wave is an alpha wave component.

14. The system of claim 13, wherein the estimated value of phase amplitude modulation is calculated based on a phase of the slow wave component and an amplitude of the alpha wave component.

15. The system of claim 11, wherein the at least one value of cross-frequency coupling comprises a correlation value between an oscillatory component included in the state space model and a band of frequencies of the EEG data.

16. The system of claim 11, wherein the fitting the state space model comprises fitting harmonic frequencies of each oscillatory component included in the at least one oscillatory component.

17. The system of claim 11, wherein the at least one oscillatory component includes an alpha component, and the alpha component is associated with prior distribution for a center frequency.

18. The system of claim 17, wherein the prior distribution is a Von Mises prior.

19. The system of claim 11, wherein a damping factor of the state space model is constrained with a prior distribution.

20. The system of claim 19, wherein the prior distribution is a beta distribution.
